(19) United States
(12) Patent Application Publication
(10) Pub. No.: US 2006/0194765 A1
(43) Pub. Date: Aug. 31, 2006

(54) Methods and compositions using oxidized phospholipids

(76) Inventors: Joe G. N. Garcia, Chicago, IL (US); Konstantin Birukov, Chicago, IL (US); Norbert Leitinger, Charlottesville, VA (US); Valery Bochkov, Vienna (AT)

Correspondence Address: EDWARDS ANGELL PALMER & DODGE LLP, P.O. BOX 55874, BOSTON, MA, 02205, US

(21) Appl. No.: 11/264,370

(22) Filed: Oct. 31, 2005

Related U.S. Application Data
(60) Provisional application No. 60/628,382, filed on Nov. 16, 2004.

(51) Int. Cl. A61K 31/685

(52) U.S. Cl. 514/78

(57) ABSTRACT

The instant invention provides compositions, e.g., compositions comprising oxidized phospholipids, for the treatment of diseases, disorders and conditions, e.g., cute lung injury syndromes, sepsis, vascular leakage, edema, acute respiratory distress syndrome (ARDS) or acute inflammation.

### RELATED APPLICATIONS

This application claims the benefit of U.S. Provisional Application No. 60/628,382, filed Nov. 16, 2004, the contents of which are hereby expressly incorporated herein by reference.

### GOVERNMENT SUPPORT

The research described herein was funded in part by grants HL 58064, HL 69340, HL 67307, HL 73994, and HL 76259 from the National Heart, Lung and Blood Institute. Accordingly, the government may have certain rights to this invention.

### BACKGROUND OF THE INVENTION

Oxidized phospholipids are biologically active components of mildly oxidized low-density lipoprotein (LDL), whose role in development of vascular injury and inflammation in systemic circulation is well recognized. Oxidized LDL is implicated in the recruitment of monocytes and foam cell formation, increased expression of matrix metalloproteinases, which is critical for both plaque formation and destabilization, proliferative response of vascular smooth muscle cells, increased thrombogenic activity of platelets, and increased endothelial-monocyte interaction.

Biologically active oxidized phospholipids derived from oxidation of 1-palmitoyl-2-arachidomoyl-sn-glycero-3-phosphorylcholine (OxPAPC) stimulate tissue factor expression, activate endothelial cells to bind monocytes, but do not cause any neutrophil binding. In addition, OxPAPC strongly inhibits LPS-mediated induction of neutrophil binding and expression of E-selectin, an adhesion molecule involved in EC inflammatory activation by endotoxin.

### SUMMARY OF THE INVENTION

The instant invention provides methods and compositions for the treatment of conditions, diseases, and disorders, e.g., acute lung injury, sepsis and acute respiratory distress syndrome (ARDS), using oxidized phospholipids, and also provides methods and compositions for the enhancement of endothelial cell barrier protective activity in a subject.

In one aspect, the invention provides a method of enhancing endothelial cell barrier protective activity in a subject by administering to a subject an effective amount of oxidized phospholipids, thereby enhancing the endothelial cell barrier protective activity in the subject.

In one embodiment, the phospholipids are phosphatidylserines, phosphatidylinositols, phosphatidylethanolamines, phosphatidylcholines or 1-Palmytoyl-2-Arachidonoyl-sn-Glycero-2-Phosphates. In another related embodiment, the phospholipids have unsaturated bonds, i.e., double bonds in the fatty acid chain of the phospholipid. In another related embodiment, the phospholipids are arachidonic acid containing phospholipids. In a specific embodiment, the phospholipids are sn-2-oxygenated. In another specific embodiment, the phospholipids are not fragmented.

In a specific embodiment, the oxidized phospholipids used in the methods and compositions of the invention are oxidized 1-palmitoyl-2-arachidonoyl-sn-glycero-3-phosphorylchloine (OxPAPC). In a related embodiment, the oxPAPCs are epoxyisoprostane-containing phospholipids.

In specific embodiments, the oxPAPC used in the methods and compositions of the invention is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

In a related embodiment, the methods of the invention are for the treatment of a subject having acute lung injury syndromes, sepsis, vascular leakage, edema, acute respiratory distress syndrome (ARDS) or acute inflammation.

In another aspect, the instant invention provides a method of enhancing endothelial cell barrier protective activity in a subject by administering to a subject an effective amount of epoxyisoprostane-containing phospholipids, thereby enhancing the endothelial cell barrier protective activity in the subject.

In a related embodiment, the epoxyisoprostane-containing phospholipids are 1-palmitoyl-2-(5,6-epoxyisoprostane E2)-sn-glycero-3-phosphocholines (5,6-PEIPC), 1palmitoyl-2-(epoxycyclopentenone)-sn-glycero-3-phosphorylcholines (PECPC) or 1-palmitoyl-2-(epoxyisoprostane E2)-sn-glycero-4-phosphocholines (PEIPC).

In another aspect, the instant invention provides a method of treating a subject having an acute lung injury by administering to a subject an effective amount of oxidized phospholipids, thereby treating the acute lung injury in the subject.

In one embodiment, the phospholipids are phosphatidylserines, phosphatidylinositols, phosphatidylethanolamines, phosphatidylcholines or 1-Palmytoyl-2-Arachidonoyl-sn-Glycero-2-Phosphates. In another related embodiment, the phospholipids have unsaturated bonds, i.e., double bonds in the fatty acid chain of the phospholipid. In another related embodiment, the phospholipids are arachidonic acid containing phospholipids. In a specific embodiment, the phospholipids are sn-2-oxygenated. In another specific embodiment, the phospholipids are not fragmented.

In a specific embodiment, the oxidized phospholipids used in the methods and compositions of the invention are oxidized 1-palmitoyl-2-arachidonoyl-sn-glycero-3-phosphorylchloine (OxPAPC). In a related embodiment, the oxPAPCs are epoxyisoprostane-containing phospholipids.

In specific embodiments, the oxPAPC used in the methods and compositions of the invention is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

In another aspect, the instant invention provides a method of treating a subject having an acute lung injury by administering to a subject an effective amount of epoxyisoprostane-containing phospholipids, thereby treating the acute lung injury in the subject.

In specific embodiments, the oxPAPC used in the methods and compositions of the invention is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

In another aspect, the instant invention provides a method of treating a subject having sepsis by administering to a subject an effective amount of oxidized phospholipids, thereby treating sepsis in the subject.

In one embodiment, the phospholipids are phosphatidylserines, phosphatidylinositols, phosphatidylethanolamines, phosphatidylcholines or 1-Palmytoyl-2-Arachidonoyl-sn-Glycero-2-Phosphates. In another related embodiment, the phospholipids have unsaturated bonds, i.e., double bonds in the fatty acid chain of the phospholipid. In another related embodiment, the phospholipids are arachidonic acid containing phospholipids. In a specific embodiment, the phospholipids are sn-2-oxygenated. In another specific embodiment, the phospholipids are not fragmented.

In a specific embodiment, the oxidized phospholipids used in the methods and compositions of the invention are oxidized 1-palmitoyl-2-arachidonoyl-sn-glycero-3-phosphorylchloine (OxPAPC). In a related embodiment, the oxPAPCs are epoxyisoprostane-containing phospholipids.

In specific embodiments, the oxPAPC used in the methods and compositions of the invention is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

In another aspect, the instant invention provides a pharmaceutical composition comprising an oxidized phospholipids and a pharmaceutically active carrier.

In a related embodiment, the phospholipids are phosphatidylserines, phosphatidylinositols, phosphatidylethanolamines, phosphatidylcholines or 1-Palmytoyl-2-Arachidonoyl-sn-Glycero-2-Phosphates. In another related embodiment, the phospholipids have unsaturated bonds, i.e., double bonds in the fatty acid chain of the phospholipid. In another related embodiment, the phospholipids are arachidonic acid containing phospholipids. In a specific embodiment, the phospholipids are sn-2-oxygenated. In another specific embodiment, the phospholipids are not fragmented.

In a specific embodiment, the oxidized phospholipids used in the methods and compositions of the invention are oxidized 1-palmitoyl-2-arachidonoyl-sn-glycero-3-phosphorylchloine (OxPAPC). In a related embodiment, the oxPAPCs are epoxyisoprostane-containing phospholipids.

In specific embodiments, the oxPAPC used in the methods and compositions of the invention is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3phosphorylcholine (PECPC) or 1-palmitoyl-2-(epoxyisoprostane E2)-sn-glycero-4-phosphocholine (PEIPC).

In another aspect, the instant invention provides a kit for the treatment of acute lung injury syndromes, sepsis, vascular leakage, edema, acute respiratory distress syndrome (ARDS) or acute inflammation comprising oxidized phospholipids and instructions for use.

In a specific embodiment, the oxidized phospholipids used in the methods and compositions of the invention are oxidized 1-palmitoyl-2-arachidonoyl-sn-glycero-3-phosphorylchloine (OxPAPC). In a related embodiment, the oxPAPCs are epoxyisoprostane-containing phospholipids.

In specific embodiments, the oxPAPC used in the methods and compositions of the invention is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

In another aspect, the instant invention provides a pharmaceutical composition comprising an oxidized phospholipids and a pharmaceutically active carrier.

In a related embodiment, the phospholipids are phosphatidylserines, phosphatidylinositols, phosphatidylethanolamines, phosphatidylcholines or 1-Palmytoyl-2-Arachidonoyl-sn-Glycero-2-Phosphates. In specific embodiments, the oxPAPC used in the methods and compositions of the invention is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

### DESCRIPTION OF THE DRAWINGS

FIGS. 1A-G depict the effects of oxidized phospholipids on transendothelial electrical resistance (TER) changes in human pulmonary endothelial cells. A—Cells were treated with 0, 5, 10, and 20 μg/ml OxPAPC. B—Effects of native PAPC on TER changes in HPAEC. Cells were treated with 0, 5, 10, and 20 μg/ml PAPC. C—Effects of OxPAPC, PAPC, PLPC, OxPLPC, and DMPC treatment on TER changes in endothelial cells. Each phospholipid was used at 20 μg/ml. In selected experiments, OxPAPC was pretreated with butylated hydroxytoluene (BHT, 5 μM, 10 min). D—Effect of OxPAPC on EC barrier recovery after thrombin stimulation. HPAEC were challenged with thrombin (50 nM) followed by OxPAPC addition (20 μg/ml) as indicated by arrows. Control cells were stimulated with thrombin alone. Shown are cumulative data from five independent experiments. E—Quantitation of OxPAPC barrier-protective effects against thrombin-induced EC barrier compromise. TER measurements at the time points indicated by dotted arrows in Panel D are expressed as % of maximal permeability in EC monolayers after 15 thrombin stimulation (50 nM, 15 min). Results are mean±SD of five independent experiments. *P<0.05. F—Concentration-dependent effects of S1P and OxPAPC on TER changes. HPAEC monolayers were treated with phospholipids at indicated concentrations, and TER were measured 15 min after stimulation. Data are presented as % of maximal TER increase. Results are mean±SD of five independent experiments. *P<0.05. G—Additive effect of OxPAPC and S1P on TER increase. HPAEC were treated with OxPAPC (20 μg/ml) and S1P (1 μM) alone, or administered together. Control cells were left untreated. Results are mean±SD of five independent experiments.

FIGS. 2A-B depict the time-dependent effects of OxPAPC on the HPAEC actin cytoskeleton. A—Cells were treated with OxPAPC (20 μg/ml) for the indicated periods of time. B—F-actin structure at the cell-cell interface of HPAEC stimulated with OxPAPC (20 μg/ml) and S1P (1 μM). OxPAPC induces unique actin microspike formation. Shown are representative results of three independent experiments. Bar=5 μm.

FIGS. 3A-B depict oxygenated, but not fragmented phospholipids exhibit barrier-protective effect. A—Mass-spectra of OxPAPC and fractions 1 and 2 obtained by preparative thin layer chromatography, as described in Materials and Methods. Arrows indicate peaks corresponding to the major phospholipid products present in fractions 1 and 2. B—Effects of OxPAPC and fractions 1 and 2 on TER. Concentrations indicated in the Figure for fractions 1 and 2 (10 μg/ml, 20 μg/ml, and 50 μg/ml) correspond to the amount of OxPAPC from which fractions 1 and 2 were obtained. OxPAPC at 100 μg/ml exhibits barrier-disruptive effect compared to prominent barrier-protective effect observed at 20 μg/ml. The results are representative of 3 experiments using 2 preparations of fractions 1 and 2. C—Effects of OxPAPC and fractions 1 and 2 on actin cytoskeleton. Cells were treated with OxPAPC, OxPAPC fraction 1, or OxPAPC fraction 2 (20 μg/ml, 20 min). F-actin was visualized by Texas Red phalloidin staining. Shown are representative results of three independent experiments. Bar=5 μm. D—Dose-dependent effects of synthetic POVPC, LysoPC and PGPC on endothelial monolayer TER. Cells were treated with indicated concentrations of synthetic phospholipids. Shown are representative results of three independent experiments.

FIGS. 4A-B depict OxPAPC activates Rac and Cdc42. A—Effect of inhibitors on OxPAPC-mediated EC barrier regulation. Cells were preincubated with the C. dificile toxin B (1 ng/ml) or Y27632 (5 μM) 30 min prior to OxPAPC (20 μg/ml) challenge. Results are expressed as percent of TER increase at 30 min in response to OxPAPC. Results are mean±SD of three independent experiments. *P<0.05. B—Effects of OxPAPC and OxPAPC Fraction #1 and Fraction #2 on Cdc42, Rac, and Rho activity. Activated GTP-bound forms of Rac, Cdc42 and Rho after OxPAPC (20 μg/ml) stimulation for indicated periods of time were isolated using pulldown assays. Effects of OxPAPC fractions equal to 20 μg/ml of OxPAPC on Rac and Rho activation (right panels) were measured after 15 min of stimulation. Total Rac, Cdc42 and Rho content in cell lysates was verified by immunoblotting. S1P (0.5 μM, 5 min) and thrombin (50 nM, 5 min) stimulation were used as positive controls for Rac and Rho activation, respectively. C—Translocation of Cdc42, Rac, and PAK, but not Rho, to the membrane/cytoskeletal fraction after OxPAPC stimulation was detected by subcellular fractionation followed by western blot analysis, as described in Materials and Methods.

FIG. 5A-B depict the effects of Rac, Cdc42, and Rho activation and inhibition on OxPAPC-mediated cytoskeletal remodeling and TER changes. A—Effects of expression of constitutively active Cdc42 (L61Cdc42), Rac (V12Rac), and Rho (V14Rho) on F-actin remodeling. Transfected cells are depicted on the lower panels. B—Co-transfection with constitutively active mutants V12Rac and L61Cdc42 (upper panels) mimics cortical F-actin rearrangement induced by OxPAPC of non-transfected cells (lower panels). High magnification insets depict actin remodeling in the cell peripheral areas. C—Effects of co-expression of dominant negative Rac (N17Rac) and Cdc42 (N17Cdc42) mutants on peripheral cytoskeletal remodeling induced by OxPAPC and Fraction #2. Cells were transfected with empty vector (lower panels) or were co-transfected with N17Rac and N17Cdc42 (upper panels) followed by stimulation with OxPAPC or Fraction #2 (20 μg/ml, 20 min, right panels). Shown are merged immunofluorescent images stained with Texas red phalloidin to visualize F-actin (red) and anti-myc tag Ab for detection of Rac/Cdc42-overexpressing cells. Insets depict magnified areas of cell-cell interface (F-actin staining in transfected cells after merging appears as yellow). Arrows point to the cortical actin band in OxPAPC-treated cells. Shown are representative results of three independent experiments. D—HPAEC grown on gold microelectrodes were incubated with siRNA to Rac1, Cdc42, Rho, or treated with non-specific RNA duplexes, as described in Materials and Methods and used for TER measurements. Cells were stimulated with OxPAPC or Fraction #2 (20 μg/ml) in the time marked by arrow. E—Cells grown in D35 culture plates were incubated with siRNA to Rac1, Cdc42, Rho, or treated with non-specific RNA duplex oligonucelotide, and target protein depletion was examined by immunobloting with corresponding antibody. Control blots represent β-actin expression in EC treated with siRNA. Shown are representative results of three independent experiments.

FIGS. 6A-D depict a molecule with m/z 810 (PECPC) co-elutes with biological activity. A—Fraction 2 obtained by preparative thin layer chromatography was further separated by reversed-phase HPLC as described in the “Methods” section. Fractions corresponding to peaks of optical density at 250 nm (line, left axis) were collected and tested for effects on TER (bars, right axis). B and C—Elution profile of PECPC and PEIPC was monitored by on-line ESI-MS at m/z values of 810.5 and 828.5, respectively. D—Mass-spectrum of the fraction eluting at 25.5 min, which demonstrated the highest TER-increasing activity.

FIG. 7 depicts the effects of OxPAPC on Raf, MEK-1,2, Erk-1,2, p90RSK, and Elk phosphorylation. HPAEC were treated with OxPAPC (20 μg/ml) or PAPC (20 μg/ml) for the indicated periods of time (left panels). On the right panels, HPAEC were pretreated for 1 hour with MEK inhibitor UO126 (5 μM), tyrosine kinase inhibitor genistein (100 μM), cell permeable PKC peptide inhibitor (20 μM), or vehicle and stimulated with OxPAPC (20 μg/ml, 15 min). Phosphorylation of MAP kinases and their downstream effectors was analyzed by immunobloting of cell lysates with a panel of phospho-specific antibodies, as described in Materials and Methods. Equal protein loadings were verified by membrane reprobing with pan-Erk-1,2 antibody. Shown are representative results of three independent experiments.

FIG. 8 depicts the effect of OxPAPC on MKK 3/6, p38, HSP-27, JNK, and ATF-1 phosphorylation. Left panel: time course of OxPAPC-mediated activation of p38- and JNK MAP kinase cascade. HPAEC were treated with OxPAPC (20 μg/ml) for the indicated periods of time. TGF-β (10 ng/ml, 30 min) was used as positive control for p38 and JNK activation. Right panels: HPAEC were incubated with OxPAPC (20 μg/ml), PAPC (20 μg/ml), or OxPAPC preincubated for 10 min with free radical blocker BHT (10 μM). Phosphorylation of MAP kinases and their downstream effectors was analyzed by immunobloting with a panel of phospho-specific antibodies, as described in Materials and Methods. Equal protein loadings were verified by membrane reprobing with pan-p38 and pan-JNK antibodies. Shown are representative results of three independent experiments.

FIGS. 9A-B depict the results indicating that OxPAPC increases protein tyrosine phosphorylation. A: time course of OxPAPC-induced protein tyrosine phosphorylation. HPAEC were treated with OxPAPC (20 μg/ml) for the indicated periods of time. B: HPAEC were pretreated for 1 hour with tyrosine kinase inhibitor genistein (100 μM), or vehicle and stimulated for 15 min with OxPAPC (20 μg/ml), PAPC (20 μg/ml), or OxPAPC preincubated for 10 min with BHT (10 μM). Total protein tyrosine phosphorylation was detected on immunoblot with anti-phosphotyrosine antibody, as described in Materials and Methods. Equal protein loadings were verified by membrane reprobing with pan-Erk-1,2 antibodies. OxPAPC induces time-dependent activation of protein tyrosine phosphorylation, which was abolished by genistein and was not affected by OxPAPC pretreatment with BHT. PAPC does not increase protein tyrosine phosphorylation. Shown are representative results of three independent experiments.

FIGS. 10A-B depict OxPAPC-induced activation of protein kinase C. A: HPAEC were treated with OxPAPC (20 μg/ml) for the indicated periods of time, and PKC-mediated phosphorylation of endogenous substrates was monitored by immunoblotting with anti-phospho-PKC substrate antibody as described in Materials and Methods. Right panel: HPAEC were pretreated with cell permeable PKC peptide inhibitor (20 μM) 1 hour prior to OxPAPC stimulation, or cells were treated with OxPAPC or PAPC (20 μM) alone. Equal protein loadings were verified by membrane reprobing with pan-Erk-1,2 antibodies. Shown are representative results of three independent experiments. B: HPAEC stimulated with OxPAPC (20 μg/ml, 15 min) were lysed, and PKC activity in cell lysates was determined in in vitro kinase assay, as described in Material and Methods. HPAEC preincubation with PKC peptide inhibitor and bisindolmaleimide I (1 μM) was performed for 1 hour prior to OxPAPC stimulation. PKC activity is expressed as pmol phosphate incorporated per mg protein per minute. Results are mean±SD of three independent experiments. *P<0.05.

FIGS. 11 A-B depict OxPAPC-induced protein kinase A activation. A: HPAEC were treated with OxPAPC (20 μg/ml) for the indicated periods of time, and PKA-mediated phosphorylation of endogenous substrates was monitored by immunoblotting with anti-phospho-PKA substrate antibody as described in Materials and Methods. Right panel: HPAEC were pretreated with cell permeable PKA peptide inhibitor (20 μM) 1 hour prior to OxPAPC stimulation, or cells were treated with OxPAPC or PAPC (20 μM) alone. Equal protein loadings were verified by membrane reprobing with pan-Erk-1,2 antibodies. Results are representative of three independent experiments. B: HPAEC stimulated with OxPAPC (20 μg/ml, 15 min) were lysed, and PKA activity in cell lysates was determined in in vitro kinase assay, as described in Material and Methods. HPAEC preincubation with PKA peptide inhibitor (20 μM) was performed for 1 hour prior to OxPAPC stimulation. PKA activity is expressed as pmol phosphate incorporated per mg protein per minute. Results are mean±SD of three independent experiments. *P<0.05.

FIG. 12 depicts the effect of OxPAPC on phosphorylation of MYPT-1, MLC, and cofillin. HPAEC were treated with OxPAPC (20 μg/ml) for the indicated periods of time, and phosphorylation of MYPT-1, MLC, and cofillin was detected by immunoblotting with corresponding phospho-specific antibody, as described in Materials and Methods. Equal protein loadings were verified by membrane reprobing with pan-MLC antibody. Shown results are representative of three independent experiments.

FIG. 13 depicts the effect of OxPAPC on phosphorylation of paxillin and FAK. Left panel: HPAEC were treated with OxPAPC (20 μg/ml) for the indicated periods of time. Right panel: HPAEC were pretreated with p60Src-specific inhibitor PP-2 (1 μM) or vehicle for 1 hour and stimulated with OxPAPC (20 μg/ml, 15 min), or treated with PAPC (20 μg/ml), or with OxPAPC preincubated for 10 min with BHT (10 μM). Phosphorylation of paxillin-Tyr¹¹⁸ and FAK-Tyr⁵⁷⁶ was detected by immunoblotting with corresponding phospho-specific antibody, as described in Materials and Methods. Equal protein loadings were verified by membrane reprobing with pan-paxillin and pan-FAK. antibodies. Shown results are representative of three independent experiments.

### DETAILED DESCRIPTION OF THE INVENTION

Increased vascular leakage is associated with numerous life threatening diseases, e.g., acute lung injury, sepsis and acute respiratory distress syndrome (ARDS). Increased lung vascular permeability results in excessive leukocyte infiltration, alveolar flooding, and pulmonary edema. The present invention is based on the discovery that oxidized phospholipids are capable of increasing endothelial cell barrier function and treatment of these conditions.

Accordingly, the invention provides methods for the treatment of subjects having, for example, acute lung injury, sepsis and acute respiratory distress syndrome (ARDS). The invention also provides methods and compositions for the enhancement of endothelial cell barrier protective activity in a subject.

Therapeutic methods of the invention can also include the step of identifying that the subject is in need of treatment of diseases or disorders described herein. The identification can be in the judgment of a subject or a health professional and can be subjective (e.g., opinion) or objective (e.g., measurable by a test or a diagnostic method). In each of these methods, a sample of biological material, such as blood, tissue, plasma, semen, or saliva, is obtained from the subject to be tested. Thus, the methods of the invention can include the step of obtaining a sample of biological material (such as a bodily fluid) from a subject; testing the sample to determine the presence or absence of a marker for a disease, disorder or condition disclosed herein; and determining whether the subject is in need of treatment according to the invention.

The methods delineated herein can further include the step of assessing or identifying the effectiveness of the treatment or prevention regimen in the subject by assessing the presence, absence, increase, or decrease of a marker. Such assessment methodologies are known in the art and can be performed by commercial diagnostic or medical organizations, laboratories, clinics, hospitals and the like. As described above, the methods can further include the step of taking a sample from the subject and analyzing that sample. The sample can be a sampling of cells, genetic material, tissue, or fluid (e.g., blood, plasma, sputum, etc.) sample. The methods can further include the step of reporting the results of such analyzing to the subject or other health care professional. The method can further include additional steps wherein (such that) the subject is treated for the indicated disease or disease symptom.

The invention provides oxidized phospholipids for the treatment of subjects having a disease or disorder disclosed herein. The phospholipids used in the method of the invention may be, for example, phosphatidylserines, phosphatidylinositols, phosphatidylethanolamines, phosphatidylcholines or 1-Palmytoyl-2-Arachidonoyl-sn-Glycero-2-Phosphates. In certain embodiments the phospholipids are arachidonic acid containing phospholipids.

In particular embodiments, the phospholipids of the invention are sn-2-oxygenatedphospholipids. In other embodiments, the phospholipids of the invention are not fragmented. In a specific embodiment, the phospholipids used in the methods of the invention are oxidized products of 1-palmitoyl-2-arachidonoyl-sn-glycero-3-phosphorylchloine (OxPAPC). In another specific embodiment, the phospholipids used in the methods of the invention are epoxyisoprostane-containing phospholipids.

“Phospholipids” are lipids that contain one or more phosphate groups. Exemplary phospholipids are phosphatidylinositol, phosphatidylserine, phosphatidylethanolamine, and phosphatidylcholine. Phospholipids are a primary component of cell membranes. In a specific embodiment of the invention, the phospholipids do not contain, and are not products of the oxidation of, 1-palmitoyl-2-arachidonoyl-sn-glycero-3-phoshorylcholine.

Phospholipids can be isolated from an organism by one of skill in the art using only routine experimentation. Moreover, phospholipids are readily available from commercial sources for purchase. For example, Sigma Aldrich (St. Louis, Mo.) sells a number of phospholipids.

Phospholipids can be oxidized by methods known by one of skill in the art. For example, as described in the Examples, phospholipids can be oxidized by exposing dry phospholipids to air for an extended period of time. Moreover, the oxidation of the phospholipids an be monitored by ESI-MS as described in the Examples.

The oxidized phospholipids used in the methods of the instant invention are sometimes referred to herein as “active ingredients”.

The term “treated,” “treating” or “treatment” includes the diminishment or alleviation of at least one symptom associated or caused by the state, disorder or disease being treated.

The term “subject” is intended to include organisms, e.g., prokaryotes and eukaryotes, which are capable of suffering from or afflicted with a condition, disease or disorder disclosed herein. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from condition, disease or disorder disclosed herein.

The language “effective amount” of the compound is that amount necessary or sufficient to treat or prevent a condition, disease or disorder described herein, e.g. acute lung injury syndromes, sepsis, vascular leakage, edema, acute respiratory distress syndrome (ARDS) or acute inflammation. The effective amount can vary depending on such factors as the size and weight of the subject, the type of illness, or the particular oxidized phospholipid. For example, the choice of the oxidized phospholipid can affect what constitutes an “effective amount”. One of ordinary skill in the art would be able to study the factors contained herein and make the determination regarding the effective amount of the oxidized phospholipid without undue experimentation.

Moreover, the compositions of the instant invention are useful in the treatment of diseases and disorders associated tissue infiltration of blood leukocytes, such as monocytes and lymphocytes. Accordinlgy, the oxidized phospholipids described herein may be effective as therapeutic agents and/or preventive agents for diseases such as atherosclerosis, asthma, pulmonary fibrosis, myocarditis, ulcerative colitis, psoriasis, asthma, ulcerative colitis, nephritis (nephropathy), multiple sclerosis, lupus, systemic lupus erythematosus, hepatitis, pancreatitis, sarcoidosis, organ transplantation, Crohn's disease, endometriosis, congestive heart failure, viral meningitis, cerebral infarction, neuropathy, Kawasaki disease, and sepsis in which tissue infiltration of blood leukocytes, such as monocytes and lymphocytes, play a major role in the initiation, progression or maintenance of the disease.

In another embodiment, the invention provides methods or monitioring the efficacy of treatment of an individual after being administered an oxidized phospholipid, e.g., the oxidized phospholipids as described herein. For example, a clinician may monitor the patient for decreased emdimas, decreases in inflammation, increased blood oxygen, increased barrier response, improvements in patient health, and or an increase in Cdc42 activation.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient; preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats a condition, disease or disorder disclosed herein.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

In yet another embodiment, the invention provides methods for testing oxidized phospholipids for the ability to treat a condition, disease or disorder as described herein. For example, one of skill in the art, could oxidize a number of phospholipids and test them for the effects as described in the examples. Moreover, one of skill in the art can separate the various oxidized phospholipids and test individual species of oxidation products for the ability to treat the conditions, diseases or disorders described herein.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Materials and Methods

Human pulmonary endothelial cells (HPAEC) were cultured and transfected with cDNAs as described previously (Birukov et al., 2002). Lipid oxidation and analysis of oxidation products by positive ion electrospray mass spectrometry (ESI-MS) was performed as described previously (Watson et al., 1997, Leitinger et al. 1999 and, Bochov et al. 2002). Measurements of transendothelial electrical resistance were performed using electrical cell substrate impedance-sensing (ECIS) system as described elsewhere (Garcia et al., 2001 and Birukova et al., 2004). Transient transfections and siRNA-based protein depletion of small GTPases were performed as described elsewhere (Birukov et al. 2002, Birukova et al., 2004 and Birukova et al., 2004a). Rac, Cdc42 and Rho activation assays were performed using assay kits from Upstate Biotechnology (Lake Placid, N.Y.) (Garcia et al., 2001 and Birukova et al., 2004). Subcellular protein fractionation, western blot analyses and densitometric analyses were performed from at least 3 experiments as described (Birukova et al., 2004). Immunofluorescent staining of HPAEC was performed as previously described (Birukov et al., 2002, Birukova et al., 2004). ANOVAs and a post hoc Student-Newman-Keuls test were used to compare the means of two or more different treatment groups. Results were expressed as the mean±SE. Differences between two groups were considered statistically significant with a value of P<0.05.

### Results

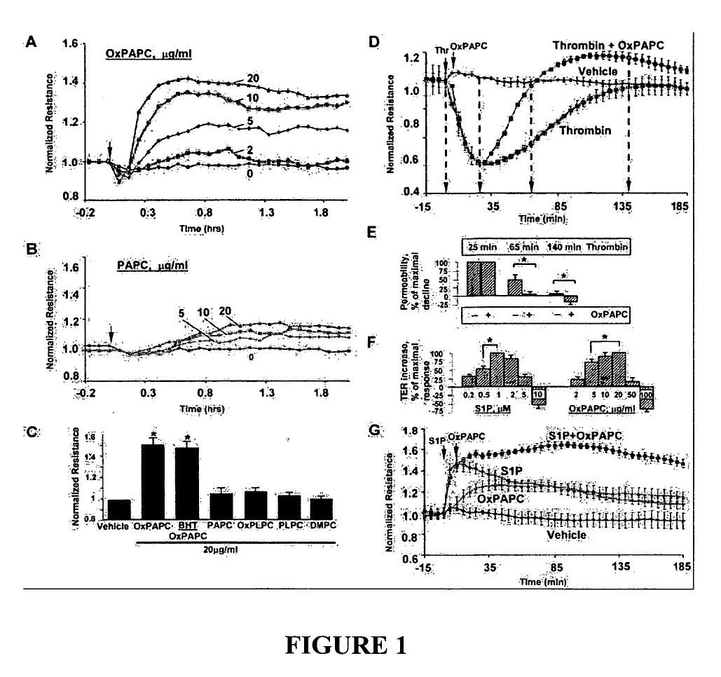

Effects of oxidized phospholipids on endothelial barrier function. OxPAPC caused dose-dependent increases in transendothelial electrical resistance (TER) across the EC monolayers with maximal response to 20 μg/ml OxPAPC (FIG. 1A,F). Barrier protective responses were dependent on oxidative modification of the PAPC, as non-oxidized PAPC or other non-oxidized phosphatidylcholines, palmitoyl-linoleate phosphatidyl choline (PLPC) and dimyristoyl phosphatidyl choline (DMPC), did not exhibit significant effects on TER, and oxidized PLPC also did not affect TER (FIG. 1B,C). Preincubation of OxPAPC with butylated hydroxytoluene (BHT) (5 μM, 10 min), a free radical quencher, prior to EC stimulation did not affect OxPAPC-induced TER increase (FIG. 1C) suggesting that the barrier-protective effect of oxidized phospholipids was not mediated by free radicals present in OxPAPC preparations.

Effects of OxPAPC on thrombin- and sphingosine 1-phosphate-induced TER changes. Thrombin treatment of pulmonary EC caused abrupt decrease in TER followed by barrier recovery. Cumulative data from five independent experiments suggest that addition of OxPAPC (20 μg/ml) to EC challenged with thrombin (50 nM) not only decreased TER recovery time more than two-fold (40 min after maximal TER decline versus 115 min with thrombin stimulation alone), but also brought TER levels above the baseline observed in non-stimulated EC (FIG. 1D,E) suggesting further barrier enhancement. Barrier-protective effects of sphingosine 1-phosphate (S1P) are mediated via G-protein-coupled Edg1 and Edg3 receptors and involve activation of small GTPase Rac¹. S1P induced rapid concentration-dependent TER increase within maximal barrier protective effect at 1 μM (FIG. 1F). OxPAPC-induced barrier-protective response reached a peak at 20 min of stimulation with maximal barrier-protective effect of OxPAPC at 20 μg/ml (FIG. 1F). Combined stimulation of pulmonary EC with OxPAPC and S1P at concentrations, which cause maximal barrier protection by each agonist alone (20 μg/ml and 1.5 μM, respectively) revealed additive effect of combined OxPAPC and S1P treatment on TER increase (FIG. 1G). These results strongly indicate distinct but additive mechanisms underlying barrier protection induced by these lipid mediators.

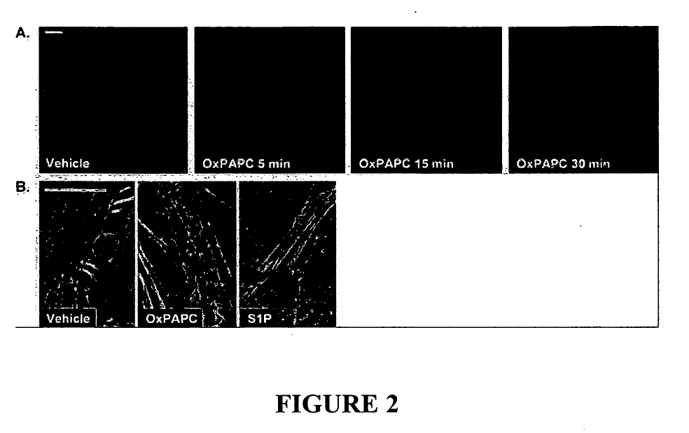

Unique EC cytoskeletal rearrangement induced by OxPAPC. Regulation of EC barrier integrity is critically dependent upon cytoskeletal elements and cell contacts (Dudek et al. 2001). OxPAPC (20 μg/ml) induced significant reduction in central F-actin stress fibers and remodeling of cortical cytoskeleton (FIG. 2A), characterized by a pronounced enhancement of peripheral F-actin staining (5 min) followed by appearance of peripheral F-actin structures (15 min), which resembled microspikes normally observed in single cells with activated small GTPases Rac and Cdc42 or PI3-kinase (Bird et al. 2003 and Levy et al., 2003). Upon completion of F-actin remodeling by 30 min of OxPAPC stimulation, HPAEC formed of a strong peripheral actin rim with disappearance of central stress fibers. Higher magnification images of cell-cell interface areas (FIG. 2B) revealed formation of unique zip-like actin projections that formed an intercollated peripheral actin cytoskeletal structures not previously observed in the S1P model of EC barrier enhancement (FIG. 2B, right panel).

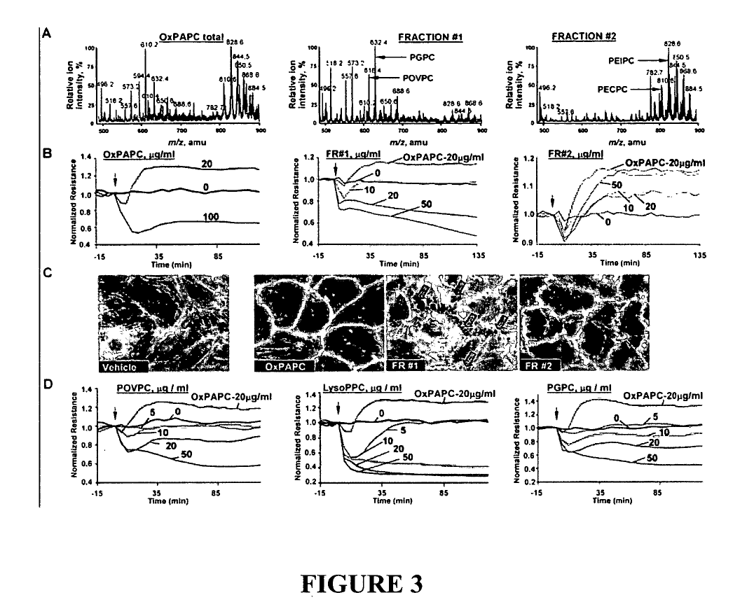

Oxygenated, but not fragmented phospholipids increase TER. In contrast to barrier protective effects exhibited by OxPAPC at 20 μg/ml, higher OxPAPC concentrations (100 μg/ml) caused barrier-disruptive effect (FIGS. 1F and 3B, left panel), which may reflect adverse effects of barrier-disruptive compounds present in OxPAPC. To further characterize biologically active molecules in OxPAPC, we separated OxPAPC by TLC into two fractions containing either fragmented (m/z <782,7, Fraction #1), or oxygenated (m/z>782,7, Fraction #2) sn-2 residues (FIG. 3A). ESI-MS-analysis demonstrated that Fraction #1 was enriched in lysoPC, POVPC and PGPG (FIG. 3A, middle panel). Fraction #1 dose-dependently decreased barrier function (FIG. 3B, middle panel). In contrast, fraction #2, which was enriched in oxygenated compounds with PEIPC and PECPC representing major peaks (FIG. 3A, right panel), induced prominent increases in TER (FIG. 3B, right panel) thus mimicking barrier protective effects of low concentrations of OxPAPC. Importantly, barrier-protective effects of fraction #2 were associated with enhancement of peripheral actin cytoskeleton also observed in OxPAPC-stimulated cells (FIG. 3C, right panel), whereas barrier-disruptive effects of fraction 1 were accompanied by gap formation, and distinct pattern of cytoskeletal remodeling with appearance of random stress fibers (FIG. 3C, middle panel). Since OxPAPC contains several oxidized phospholipids bearing a fragmented acyl chain at the sn-2 position, such as POVPC, PGPC, and lysoPC, and they are all present in OxPAPC (Watson et al., 1997, Leitinger et al., 1999 and Subbanagounder et al., 2000), we next tested effects of synthetic POVPC, lysoPC and PGPC on EC barrier properties. All three compounds, POVPC, PGPC and lysoPC, prepared by chemical synthesis significantly and concentration-dependently decreased TER (FIG. 3D). These results clearly demonstrate barrier-disruptive effects of fragmented oxidation products and lysoPC on the pulmonary EC monolayers.

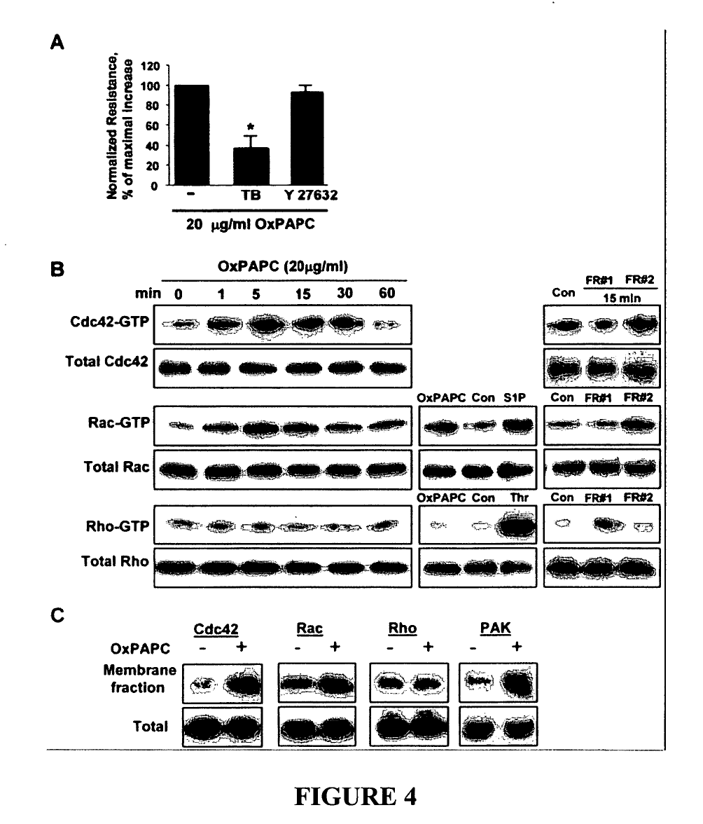

Effects of OxPAPC on activation of small GTPases Rac, Rho, and Cdc42. Previous studies have stressed out a critical role for Rho and Rac in specific cytoskeletal responses associated with endothelial barrier regulation (Garcia et al., 2001, Birukova et al., 2004 and van Nieuw Amerongen et al., 2000). FIG. 4A shows that OxPAPC-induced increases in TER were attenuated by inhibition of Rac, Cdc42 and Rho activities using toxin B (100 ng/ml), but not by HPAEC pretreatment with Rho-kinase inhibitor Y27632 (5 μM, 1 hr). These results strongly suggest an involvement of Rac and Cdc42, but not Rho in the barrier protective effects of oxidized phospholipids. Measurements of OxPAPC-induced small GTPase activation (FIG. 4B) revealed transient activation of Rac with peak at 5 min and a decline after 15 min. Furthermore, OxPAPC-induced Cdc42 activation reached a peak at 5 min and remained elevated above the basal level until 30 min of stimulation. In contrast, Rho activity was not affected by OxPAPC (FIG. 4B, lower panels). Importantly, HPAEC stimulation with OxPAPC Fraction #2, which exhibited barrier-protective properties (FIG. 3B, right panels) induced Rac and Cdc42 activation without effects on Rho activity, whereas OxPAPC Fraction #1, which contained fragmented phospholipids and did not reveal barrier-protective properties showed no significant Rac and Cdc42 activation (FIG. 4B, right panels). Subcellular fractionation studies indicated OxPAPC-induced translocation of Cdc42, Rac, and the Rac effector PAK1 (αPAK) from the cytosol to the membrane (FIG. 4C), whereas intracellular distribution of Rho remained unchanged.

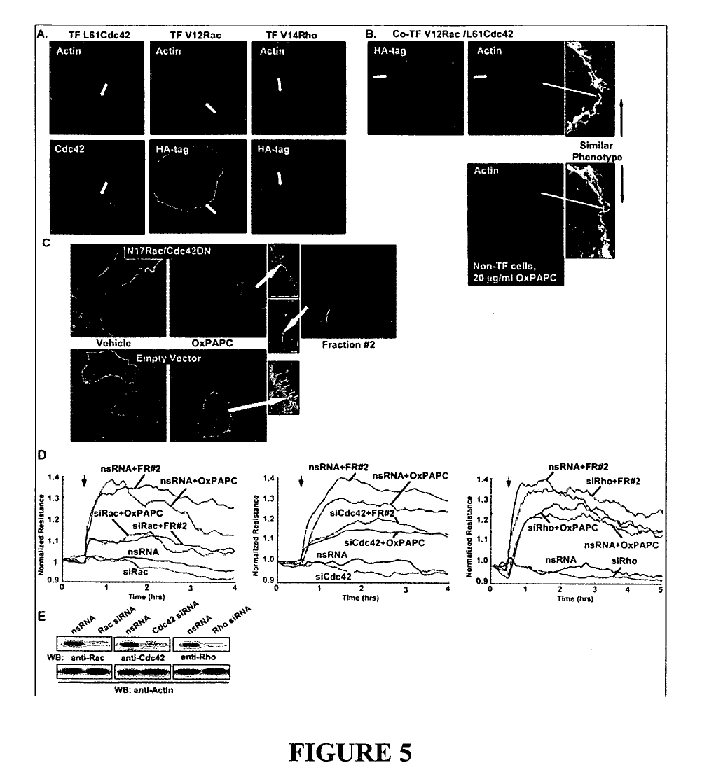

Effects of Rac and Cdc42 activities on OxPAPC-induced cytoskeletal remodeling. To test a role of coordinated Rac and Cdc42 activation in the unique cytoskeletal remodeling observed in OxPAPC-stimulated cells, HPAEC were transiently transfected with constitutively active or dominant negative Rac and Cdc42 mutants. Expression of constitutively active L61Cdc42 caused significant filopodia formation and cell retraction, while expression of constitutively active V12Rac stimulated cell spreading and enhanced cortical actin rim formation (FIG. 5A). Expression of V14Rho caused intense central stress fiber formation, the cytoskeletal effect distinct from the pattern of OxPAPC-induced actin remodeling (FIG. 5A). Because the unique OxPAPC-induced peripheral cytoskeletal remodeling was associated with activation of both Rac and Cdc42, EC were next co-transfected with V12Rac and L61Cdc42. Co-expression of activated Rac and Cdc42 induced peripheral actin cytoskeletal remodeling that resembled OxPAPC-induced effects (FIG. 5B). Finally, co-transfection of human pulmonary EC with dominant negative N17Rac and N17Cdc42 mutants completely abolished enhancement of peripheral actin cytoskeleton induced by OxPAPC or its barrier-protective Fraction #2 (FIG. 5C, upper panels), as compared to OxPAPC-stimulated cells transfected with empty vector (FIG. 5C, lower panels). HPAEC transfection with dominant negative Rac abolished OxPAPC-induced enhancement of continuous peripheral F-actin staining observed in non-transfected cells, but did not affect formation of microspike-like structures. Importantly, S1P stimulation of HPAEC overexpressing dominant negative Rac did not reveal formation of microspike-like structures observed in OxPAPC stimulated cells, again suggesting that Cdc42 activation is unique to OxPAPC-stimulated endothelial cells. We next tested effects of specific small GTPase depletion on OxPAPC-induced TER changes using siRNA-mediated knockdown of Rac, Cdc42 or Rho. Depletion of Rac and Cdc42 protein expression significantly attenuated TER increase induced by OxPAPC and TLC Fraction #2 (FIG. 5D), whereas depletion of Rho or treatment with non-specific RNA duplex oligonucleotide were without effect. Depletion of target proteins upon treatment with corresponding siRNA was confirmed by immunoblotting with appropriate antibody (FIG. 5E). Cell treatment with non-specific RNA duplex oligonucleotide did not affect small GTPase expression.

Increased phosphorylation of Rac-dependent regulator of actin polymerization cofilin stimulates peripheral actin polymerization and can be induced by OxPAPC and S1P (Garcia et al., 2001 and Bochokov et al., 2004). OxPAPC stimulation of EC monolayers induced peripheral translocation of the regulators of actin polymerization preferentially activated by Rac (cortactin, p21Arc), Cdc42 (N-WASP), and Rac/Cdc42 (Arp3, phospho-cofilin). Subcellular fractionation and western blot analysis validated the results of immunofluorescent analysis with membrane translocation of cortactin, p21Arc, Arp3, N-WASP, and phospho-cofilin in response to OxPAPC stimulation. Taken together, these data demonstrate essential role for Cdc42- and Rac-mediated signaling pathways in OxPAPC-induced endothelial barrier regulation and unique cytoskeletal remodeling driven by Rac/Cdc42 cytoskeletal effector proteins.

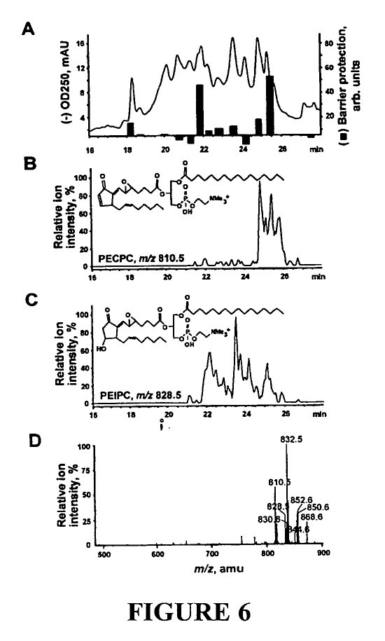

A molecule with m/z 810 (PECPC) co-elutes with biological activity in HPLC-MS. Among oxygenated derivatives of PAPC, PEIPC (m/z 828) and PECPC (m/z 810) have been structurally identified and shown to exert biological activities (Watson et al., 1997, Leitinger et al., 1999, and Subbangounder et al., 2000). Since TER-increasing activity is present in the fraction containing oxygenated PCs, we further separated the TLC fraction 2 using reversed phase HPLC-MS, which separates these compounds into several isomers (Watson et al., 1997), and tested effects of individual fractions on EC barrier properties. We found three major fractions with barrier protective activities eluted at 18 min, 21.5 min and 25.5 min (FIG. 6A). Single ion tracing for PEIPC and PECPC (m/z 810 and 828, respectively) revealed that the molecule with m/z 810 co-eluted with the fraction exhibiting major barrier-protective activity (25.5 minutes) (FIGS. 6B and 6C). ESI-MS analysis of this fraction demonstrated that PECPC (m/z 810.5, [M+Na⁺] 832.5) was the major component of this fraction, while minor components (m/z 828, 830, 844) were also present (FIG. 6D).

### Discussion

Precise regulation of endothelial semiselective barrier is critically important for mass transport and metabolic exchange between blood and peripheral tissue. Edemagenic and pro-inflammatory agents including thrombin and cytokines compromise endothelial barrier leading to extravasation of fluid and blood cells, which is a hallmark of inflammation and edema formation. In contrast to mechanisms involved in barrier dysfunction, mechanisms of EC barrier recovery are not well understood. In addition, little is known about bioactive compounds that are released during injury or inflammation and promote resealing of the endothelial monolayer, which is an important aspect in resolution of inflammation.

Our results show that specific phospholipid oxidation products induce concentration-dependent and sustained barrier-protective effects (FIGS. 1, 3 and 6), counteracting thrombin-induced EC barrier disruption (FIG. 1). These effects were specific for oxidized forms of phospholipids, since non-oxidized phospholipids in the same concentration range did not significantly affect EC permeability (FIG. 1). Structure-function analysis revealed that the barrier protective effect was independent of the phospholipid head group, since oxidized phosphatidylserine, -ethanolamine, and phosphatidic acid also increased TER. Oxidation products of arachidonic acid-, but not linoleic acid-containing phospholipids exhibited barrier-protective properties (FIG. 1), and we show that sn-2-oxygenated, but not sn-2-fragmented phospholipids, are responsible for the induction of barrier protective effects (FIG. 3). Analysis of these oxygenated products using HPLC-MS revealed that a molecule with m/z 810 corresponding to 1-palmitoyl-2-(epoxycyclopentenone)-sn-glycero-3-phosphorylcholine (PECPC) 14 and a molecule with m/z 828 corresponding to another epoxyisoprostane-containing phospholipid, 1-palmitoyl-2-(epoxyisoprostane E2)-sn-glycero-3-phosphocholine (PEIPC), co-eluted with TER increasing activity (FIG. 6). Along with PECPC and PEIPC, several other not yet identified compounds that are present in the oxygenated fraction of OxPAPC may contribute to the overall barrier protective effect (FIG. 6). It will be the goal of future studies to identify the chemical structures of these compounds.

Oxidized lipids appear in several lung disorders. For example, in acute lung injury there is leakage of native lipoproteins from serum into the alveolar space where they are oxidatively modified. Oxidative stress, intrinsic to lung injury, results from impaired antioxidant defense, the presence of reactive oxidant species, and exposure to hyperoxia during mechanical ventilation or exposure to ozone (Uhlson et al., 2002). Increased levels of oxidized phospholipids have been shown in murine lung tissue (Nakamura et al. 1998) and may also appear in lung circulation in pathological settings of acute injury, sepsis, and inflammation, all of which are also associated with platelet activation and increased release of S1 P by platelets. Our data demonstrate additive effects of oxidized phospholipids and S1P on EC barrier protection (FIG. 1). Importantly, OxPAPC and S1P trigger distinct intracellular signaling pathways with preferential activation of Cdc42 and Rac-mediated signaling and cytoskeletal remodeling by OxPAPC and Rho and Rac-mediated signaling by S1P (Garcia et al., 2001 and Shikata et al., 2003).

Although the kinetics of OxPAPC-mediated intracellular signaling (Huber et al., 2002, Bochkov et al., 2002, Birukov et al., 2004, Bochkov et al., 2002b, and Leitinger et al., 1997), cytoskeletal remodeling and barrier regulation (FIGS. 1,2) suggest a receptor-mediated cellular response, a specific receptor for OxPAPC has not yet been identified. While some specific effects of OxPAPC can be partially inhibited by platelet activating factor (PAF) receptor antagonists (Leitinger et al., 1997, Subbanagounder et al., 1999 and Kadl et al., 2002), PAF itself does not mimic barrier-protective OxPAPC effects, and instead is a well recognized edemagenic agent (Goggel et al. 2004). These observations suggest a potential structural homology of a putative OxPAPC receptor with the PAF receptor and do not exclude the potential for several receptors capable of binding different components of OxPAPC and triggering OxPAPC-mediated signal transduction Leitinger et al., 1999).

Coordinated remodeling of the actin cytoskeleton, focal adhesions and adherens junctions is precisely controlled by small GTPases (Kaibuchi et al., 1999, Turner, 2000, and Kaibuchi et al., 1999b). Activated Rho, Rac, and Cdc42 induce the formation of stress fibers, lamellipodia and filopodia, respectively (Ridley, 2001). While Rho functions mostly by reorganizing preexisting actin filaments, Rac and Cdc42 promote new actin polymerization at the cell cortical layer, either by stimulating the uncapping or severing of actin filaments (Machesky et al., 1999). Our results demonstrate for the first time that OxPAPC induces specific activation of Rac- and Cdc42 (FIG. 4), which govern a unique cytoskeletal rearrangement (FIGS. 2 and 3) characterized by an enhanced peripheral actin cytoskeleton and formation of F-actin structures at the cell-cell interface that resemble microspikes in single cells with activated Rac/Cdc42 cascade (Bird et al., 2003). These cytoskeletal changes were linked to the accumulation of Arp3, p21-Arc, cortactin, N-WASP and phospho-cofilin in the cortical layer. While activated Rac promotes lamellipodia formation via local activation of Arp2/3-cortactin-dependent actin polymerization (Borisy et al., 2000 and Weed et al., 2001) and formation of novel focal adhesion contacts, which involves PAK, GIT2, and paxillin (Turner et al., 2001), activated Cdc42 triggers N-WASP-induced filopodia and microspike formation, as well as assembly of paxillin-PAK-GIT1-GIT2 focal adhesion protein complexes Kaibuchi et al., 1999, Ridley, 2001 and Turner et al., 2001). Moreover, Cdc42 and Rac control cadherin-mediated cell-cell adhesion and formation of novel adherens junction complexes via modulation of interactions between alpha-catenin and cadherin-catenin complex (Kaibuchi et al., 1999b). Activation of both Rac and Cdc42 is involved in cell spreading after adhesion to thrombospondin-1 (Adams et al., 2000). Thus, the specific cytoskeletal rearrangement induced by OxPAPC may well be a result of combined activation of Rac and Cdc42.

An essential role for the combined Rac and Cdc42 activation in OxPAPC-mediated cytoskeletal remodeling was further supported by our results showing that only the co-expression of constitutively active Rac and Cdc42 induced the unique cytoskeletal rearrangement that was observed in OxPAPC-stimulated EC monolayers (FIG. 5) and which was different from S1P-induced actin remodeling (FIG. 2B). Moreover, co-expression of dominant negative Rac and Cdc42 abolished peripheral actin cytoskeletal remodeling induced by OxPAPC, and siRNA-based depletion of endogenous Rac and Cdc42 pools attenuated EC barrier-protective response induced by OxPAPC and its barrier-protective Fraction #2 containing oxygenated phospholipids PECPC and PEIPC (FIGS. 5,6). Taken together, these data suggest that Rac and Cdc42 serves as integrating signaling systems that mediate specific rearrangements of actin cytoskeleton and cell contacts leading to OxPAPC-mediated barrier protection in endothelial monolayers.

Based on these studies, we propose a role for oxidized phospholipids in resolution of acute inflammation involving vascular leakage. Excessive accumulation of short chain oxidized phospholipids is associated with early stages of acute lung injury characterized by high levels of oxidative stress and may compromise EC barrier function thus contributing to edema formation. However, at later phases diminished oxidative stress in the areas of tissue injury leads to the formation of oxygenated phospholipids to the levels that would enhance EC barrier function, which would represent a feedback mechanism leading to EC barrier recovery. This protective effect can be further potentiated by S1P generated by activated platelets, which acts in additive fashion with oxidized phospholipids. These findings suggest the use of controlled administration of exogenous barrier-protective oxidized phospholipids as a new therapeutic approach in the treatment of acute lung injury syndromes.

In summary, our results demonstrate for the first time barrier-protective properties of biologically active oxidized phospholipids in endothelial cells. We show that OxPAPC-induced barrier protection involves a unique cytoskeletal remodeling mediated by combined activation of the small GTPases Cdc42 and Rac. The characterization of structurally defined components of OxPAPC with the potent barrier protective effects forms a basis for targeted drug design of a novel class of anti-edemagenic and anti-inflammatory therapeutic agents and provides new insights into the role of oxidized phospholipids in the compensatory mechanisms of endothelial barrier protection under life-threatening conditions, such as acute lung injury and inflammation.

### Example 2

### Materials and Methods

Materials. All biochemical reagents including mouse monoclonal pan-MLC antibody and 1-palmitoyl-2-arachidomoyl-sn-glycero-3-phosphorylcholine (OxPAPC) were obtained from Sigma Chemical (St. Louis, Mo.) unless otherwise indicated. Rabbit polyclonal phospho-Raf, phospho-MEKK1/2, phospho-Erk-1,2, phospho-Elk, phospho-p90RSK, phospho-MKK4, phospho-p38, pan-p38, phospho-HSP-27, phospho-LNK, phospho-ATF-2, phospho-MLC, and phospho-paxillin antibodies, phospho-PKA substrate antibody, phospho-PKC substrate antibody, as well as MEK inhibitor UO126 were obtained from Cell Signalling (Beverly, Mass.). Rabbit polyclonal phospho-FAK and phospho-MYPT1 antibodies were obtained from Upstate Biotechnology (Lake Placid, N.Y.). Cell permeable PKA peptide inhibitor, PP-2, genistein and bisindolmaleimide I were purchased from Calbiochem (La Jolla, Calif.). Cell permeable PKC peptide inhibitor was obtained from Promega (Madison, Wis.), rabbit polyclonal phospho-cofillin and pan-Erk1,2 antibodies were obtained from Santa Cruz (Santa Cruz, Calif.). Mouse monoclonal anti-FAK and anti-paxillin antibodies were obtained from BD Pharmingen (San Diego, Calif.).

Cell culture. Human pulmonary artery endothelial cells were obtained from Clonetics, BioWhittaker Inc. (Frederick, Md.). Cells were maintained in complete culture medium consisting of Clonetics EBM basic medium containing 10% bovine serum and supplemented with a set of non-essential amino acids, endothelial cell growth factors, and 100 units/ml penicillin/streptomycin provided by Clonetics, BioWhittaker Inc., and incubated at 37° C. in humidified 5% CO₂ incubator. Cells were used for experiments at passages 6-8.

Lipid oxidation and analysis. PAPC was oxidized by exposure of dry lipid to air for 72 hours. The extent of oxidation was monitored by positive ion electrospray mass spectrometry (ESI-MS) as described previously (Watson et al., 1997). Lipids were stored at −70° C. in chloroform and used within 2 weeks after mass spectrometry testing. PAPC and OxPAPC preparations were shown negative for endotoxin by the limulus amebocyte assay (BioWhittaker, Frederick, Md.).

Western immunoblotting. Protein extracts were separated by SDS-PAGE, transferred to polyvinylidene difluoride (PVDF) membranes (30 V for 18 h or 90 V for 2 h), and the membranes were incubated with specific antibodies of interest. Equal protein loadings were verified by reprobing membranes with anti-Erk, anti-Fax, or anti-paxillin antibodies. Immunoreactive proteins were detected with the enhanced chemiluminescent detection system (ECL) according to the manufacturer's directions (New England BioLabs, Beverly, Mass.). The relative intensities of the protein bands were quantified by scanning densitometry using Image Quant 5.2 (Molecular Dynamics, Piscataway, N.J.) software.

Activation of MAP kinase pathways and characterization of tyrosine phosphorylation. Activation of MAP kinase cascade was by monitored by western immunoblotting techniques using phosphospecific antibodies, which are described in Materials section and detect activated form of protein kinases of the MAP kinase cascade. Analysis of the total protein tyrosine phosphorylation was performed by immunoblotting with phosphotyrosine antibody.

Analysis of PKC and PKA activities. After stimulation with OxPAPC (20 μg/ml, 15 min), HPAEC were lysed, cell lysates were clarified by centrifugation (14000 g, 5 min, +4° C.), and PKA and PKC activities were measured using in vitro kinase assay kits obtained from Promega Corp. (Madison, Wis.) according to manufacturer protocol. Additionally, OxPAPC-induced PKC and PKA activation in HPAEC cultures was determined by immunoblotting of whole cell lysates with phospho-PKC substrate- and phospho-PKA substrate-specific antibodies that recognize PKA- or PKC-phosphorylated sites in the EC endogenous proteins.

Statistical analysis. ANOVAs with a Student-Newman-Keuls test were used to compare the means of two or more different treatment groups. Results are expressed as means±SE. Differences between two groups were considered statistically significant when P<0.05.

### Results

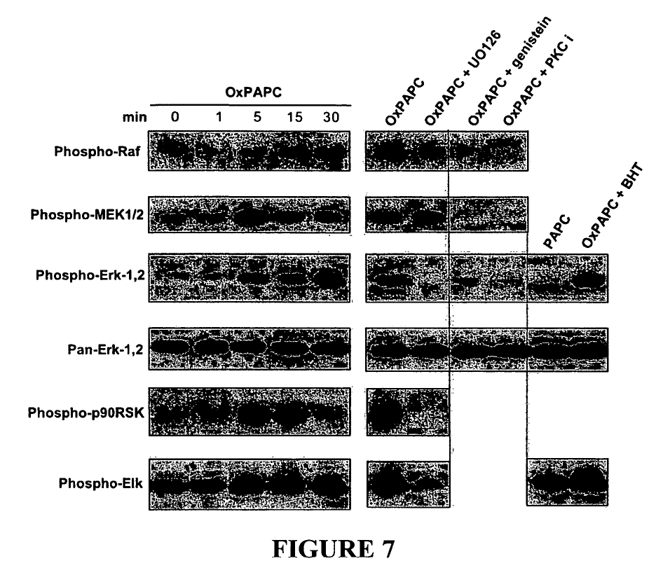

OxPAPC induces activation of MAP-kinase cascade. Stimulation of HPAEC with OxPAPC (20 μg/ml) induced time-dependent activation of Erk-1,2, which peaked at 15 min and remained elevated after 30 min (FIG. 7, left panel) and 1 hour. Erk-1,2 activation by OxPAPC was associated with activation of Erk-1,2 upstream activators MEK1,2 and Raf (FIG. 7, left panel). Erk-1,2 activation resulted in phosphorylation of its downstream targets, p90RSK and Elk. Specific MEK1,2 inhibitor, UO-126 (5 μM) completely abolished OxPAPC-induced Erk-1,2, p90Rsk, and Elk phosphorylation (FIG. 7, right panel). Broad tyrosine kinase inhibitor, genistein (100 μM), and cell permeable peptide inhibitor of PKC (20 μM) attenuated OxPAPC-induced activation of Raf, MEK 1,2, and Erk 1,2, suggesting a role for PKC and tyrosine kinases in upstream activation of MAP cascade induced by OxPAPC. Activation of MAP kinase cascade was specific for OxPAPC; as non-oxidized PAPC had no effect on Erk-1,2 activation (FIG. 1, right panel). In addition, OxPAPC preincubation with BHT, a free radical quencher, caused same levels of Erk-1,2 activation and Elk phosphorylation, as non-treated OxPAPC (FIG. 7, right panel), suggesting that effects of OxPAPC on Erk-1,2 activation are not due to residual reactive oxygen species present in OxPAPC preparation.

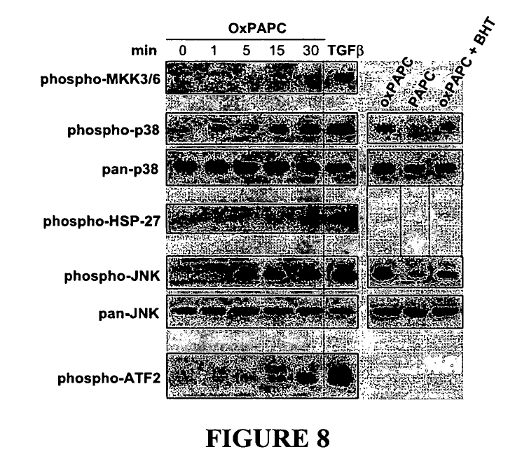

Effects of OxPAPC on p38 and JNK MAP kinases. In contrast to activation of Erk-1,2 cascade, OxPAPC did not significantly increase phosphorylation of p38 and p38-specific downstream target, HSP-27 (FIG. 8, left panel). Consistent with these observations, OxPAPC did not affect p38 upstream activator, MKK 3/6. Analysis of JNK MAP-kinase showed that OxPAPC induced phosphorylation of JNK and its downstream effector, ATF-1 (FIG. 8). OxPAPC preincubation with BHT caused same levels of JNK activation, as non-treated OxPAPC. Finally, non-oxidized PAPC was without effect on p38 and JNK MAP kinase activation (FIG. 8, right panels). Probing membranes with pan-JNK antibody showed equal JNK content in HPAEC lysates. Stimulation of HPAEC with transforming growth factor-β (TGF-β), a known activator of p38 and JNK pathways, was used as positive control in these experiments.

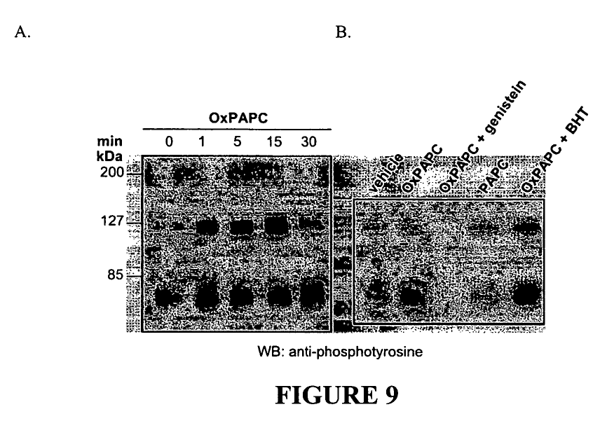

Activation of tyrosine phosphorylation in HPAEC by OxPAPC. Western blot analysis of HPAEC treated with OxPAPC showed time-dependent activation of protein tyrosine phosphorylation which peaked at 15 min and still remained elevated after 30 min of treatment (FIG. 9) and 1 hour. This activation was abolished by a broad tyrosine kinase inhibitor, genistein (100 μM) (FIG. 9, right lane). Non-oxidized PAPC was without effect on protein tyrosine phosphorylation. OxPAPC preincubation with BHT did not affect OxPAPC stimulatory effect on protein tyrosine phosphorylation (FIG. 9, right panel).

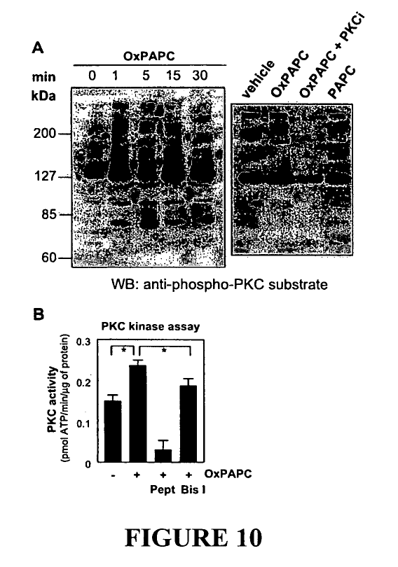

OxPAPC-induced PKC activation. Activation of PKC in HPAEC stimulated with OxPAPC was assessed using two approaches. In one series of experiments, PKC-mediated phosphorylation of endogenous protein substrates was detected by immunoblotting of HPAEC lysates with phospho-specific antibodies to PKC phosphorylation sites after OxPAPC stimulation, as described in Materials and Methods. FIG. 10A depicts a profile of endogenous PKC-mediated protein serine/threonine phosphorylation in HPAEC and demonstrates that OxPAPC challenge induced PKC-dependent phosphorylation of a broad range of endogenous substrates with major phosphorylated proteins in the 200-240 kDa, 160 kDa, 120-130 kDa, and 70-90 kDa range. PKC activation was observed after 5 min of stimulation, peaked at 15 min, and remained elevated after 30 min of stimulation. Non-oxidized PAPC did not significantly increase endogenous protein phosphorylation (FIG. 10A, right panel). Cell permeable specific PKC peptide inhibitor abolished OxPAPC-induced phosphorylation, thus confirming specificity of antibodies used for detection of PKC-mediated endogenous phosphorylation (FIG. 10A, right panel). Direct analysis of PKC activation in OxPAPC-stimulated HPAEC was performed in in vitro kinase assay with exogenous PKC-specific substrate peptide, as described in Materials and Methods. Treatment of HPAEC with OxPAPC (20 μg/ml, 15 min) significant increase in PKC activity, which was attenuated by PKC peptide inhibitor (FIG. 10B). PKC inhibitor bisindolmaleimide I attenuated OxPAPC-induced PKC activation to a lesser extent.

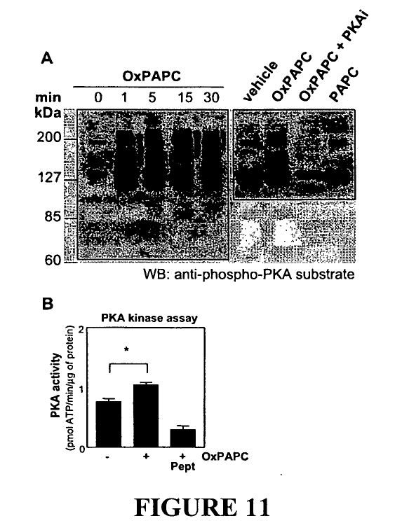

OxPAPC-induced PKA activation. Similar to analysis of PKC activation, assessment of PKA activity in HPAEC upon OxPAPC stimulation was performed by western blot with antibodies specific to PKA phosphorylation sites, and in in vitro kinase assays. FIG. 11A depicts a profile of endogenous PKA-mediated protein serine/threonine phosphorylation in HPAEC and demonstrates that OxPAPC challenge induced PKA-dependent phosphorylation of a broad range of endogenous substrates with major phosphorylated proteins in the 200-220 kDa, 140-160 kDa, 130 kDa, and 80-90 kDa range. PKA activation was observed after 5 min of stimulation, peaked at 15 min, and remained elevated after 30 min of stimulation. Cell permeable specific PKA peptide inhibitor abolished OxPAPC-induced phosphorylation, thus confirming specificity of antibodies used for detection of PKA-mediated endogenous phosphorylation (FIG. 11A, right panel). In vitro PKA kinase assay showed that OxPAPC also increased PKA activity, which was attenuated by cell permeable PKA peptide inhibitor (FIG. 11B). Non-oxidized PAPC did not induce PKA activation (FIG. 11A, right panel).

Effects of OxPAPC on cytoskeletal proteins. OxPAPC-mediated activation of PKC and tyrosine phosphorylation may induce changes in cytoskeletal organization and cell contact arrangement. In the next series of experiments, we examined effects of OxPAPC on potential cytoskeletal and cell adhesion protein targets.

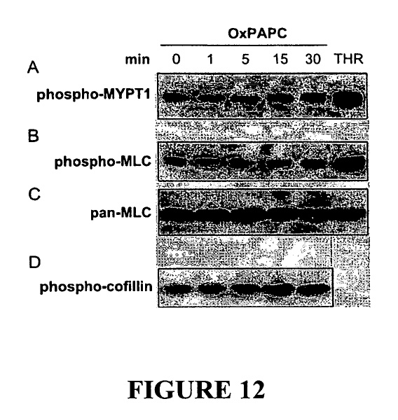

Phosphorylation of regulatory myosin light chains triggers actin stress fiber assembly, cytoskeletal rearrangement, actomyosin contraction, and may lead to endothelial cell retraction and gap formation (for review see (Dudek and Garcia, 2001)). Along with MLC kinases, myosin-specific phosphatase (MYPT1) plays a critical role in regulation of MLC phosphorylation status. Phosphorylation of Thr⁶⁸⁶ and Thr⁸⁵⁰ leads to MYPT1 inactivation and thus increases MLC phosphorylation (Carbajal et al., 2000; Velasco et al., 2002). OxPAPC treatment did not affect MLC phosphoryaltion levels, as detected by western blot with anti-diphospho-MLC antibody raised against MLC epitope containing phospho-Ser¹⁹ and phospho-Thr¹⁸ (FIG. 12, Panel B). Panel C depicts equal MLC content in the samples. OxPAPC also did not affect MYPT site-specific phosphorylation, as examined by immunobloting HPAEC lysates with a blend of MYPT anti-Thr⁶⁸⁶ and anti-Thr⁸⁵⁰ antibodies (FIG. 12, Panel A). However, OxPAPC treatment induced significant phosphorylation of cofilin, an actin-binding protein involved in regulation of actin polymerization (FIG. 12, Panel D).

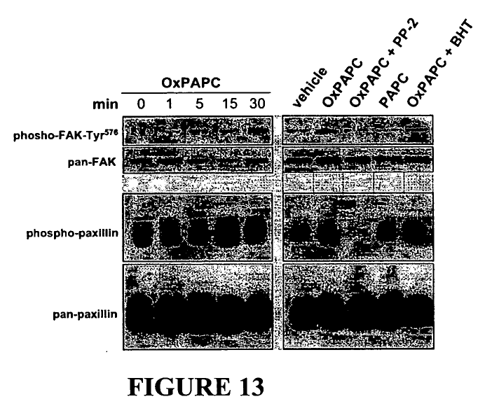

Effects of OxPAPC on FAK and paxillin phosphorylation. FAK and paxillin are focal adhesion proteins involved in cell motility and focal adhesion remodeling (Parsons et al., 2000; Turner, 2000). OxPAPC treatment induced time-dependent tyrosine phosphorylation of FAK at Tyr⁵⁷⁶, a site critical for activation of FAK catalytic activity (Parsons et al., 2000), and paxillin at Tyr¹¹⁸, the site of phosphorylation by FAK (Turner, 2000) (FIG. 13). Equal FAK and paxillin loadings were verified with pan-FAK and pan-paxillin antibodies. OxPAPC-induced phosphorylation of FAK and paxillin was attenuated by HPAEC pretreatment with p60Src-specific inhibitor PP-2 (5 μM) prior to OxPAPC stimulation (FIG. 13, right panel).

### Discussion

Oxidized LDL induce diverse physiological responses in vascular smooth muscle and endothelial cells, which include activation of cell proliferation, expression of inflammatory adhesion molecules, activation of actomyosin contraction, or activation of apoptosis (Essler et al., 1999; Leitinger et al., 1999; Li et al., 1998; Mine et al., 2002; Napoli et al., 2000; Yang-et al. 2001). Apparent inconsistency of cellular responses induced by oxidized LDL may be due to heterogeneity of LDL components (Leitinger et al., 1999; Watson et al., 1997), different LDL oxidation conditions used by investigators, and by cell type specificity of responses (Li et al., 1998; Yang et al., 2001).

OxPAPC is a bioactive component of OxLDL and oxidized cell membranes with well characterized chemical properties (Watson et al., 1997). OxPAPC induces monocyte adhesion to vascular endothelium from systemic circulation and exhibits antagonistic effect on expression of pro-inflammatory surface receptors (VCAM and E-selectin) and adhesion of neutrophils to endothelial cells induced by LPS (Bochkov et al., 2002a; Leitinger et al., 1999). Inhibitory analysis of signaling pathways triggered by OxPAPC linked physiological effects of OxPAPC to several signaling molecules such as protein kinase A (Leitinger et al., 1999), protein kinase C, and Erk-1,2 (Bochkov et al., 2002b). However, precise mechanisms of OxPAPC-mediated intracellular signaling have not been yet investigated. In this study, we characterized effects of OxPAPC on intracellular signaling in human pulmonary endothelial cells. Our results suggest a rapid activation of PKC, PKA, protein tyrosine phosphorylation and MAP kinase cascade by OxPAPC. Moreover, inhibition of PKC and tyrosine kinase activities attenuated activation of Raf, MEK-1,2, and Erk-1,2. One potential PKC-dependent mechanism involves PKC-mediated inactivation of Ras GTPase activating protein (Ras GAP) which is negative regulator of GTPase Ras, which in turn activates Raf (Gutkind, 1998). Tyrosine phosphorylation may play a role in OxPAPC-induced activation of Raf via p60Src-mediated mechanisms (Luttrell et al., 1999; Porter and Vaillancourt, 1998). OxPAPC did not activate p38 MAP kinase cascade, but modestly activated JNK and induced phosphorylation of ATF-2. Although ATF-1 is a substrate for both, p38 and JNK MAP-kinases, its phosphorylation upon OxPAPC treatment is most likely attributed to JNK activation. Differential activation of MAP kinase cascades is consistent with previous findings suggesting Erk-1,2-dependent mechanisms for activation of Egr and tissue factor expression observed in endothelial cells from systemic circulation (Bochkov et al., 2002b). Results of this study demonstrate OxPAPC-mediated activation of Erk-1,2 substrates, p90RSK and Elk involved in transcriptional regulation, and suggest a potential role for JNK effector ATF-2 in OxPAPC-induced specific gene expression in human pulmonary EC.

Activation of PKA and PKC in OxPAPC-stimulated pulmonary EC may dually impact cell function. Increased intracellular cAMP levels and consequent activation of cAMP-dependent protein kinase (PKA) exhibit protective effect on vascular leak induced by inflammatory mediators, such as thrombin, phorbol myristoyl acetate (PMA), Pertussis toxin and bacterial wall lipopolysacharide (LPS) (Adkins et al., 1993; Chetham et al., 1997; Essler et al., 2000; Garcia et al., 1995; Liu et al., 2001; Patterson et al., 1994; Patterson et al., 2000). Molecular mechanisms of barrier protective effects of PKA include: 1) PKA-mediated phosphorylation of endothelial myosin light chain kinase (MLCK) and attenuation of its activity leading to decreased basal level MLC phosphorylation (Garcia et al., 1995; Garcia et al., 1997); 2) phosphorylation of actin-binding proteins, filamin, adductin, and dematin (Hastie et al., 1997; Matsuoka et al., 1996; Wallach et al., 1978), and focal adhesion proteins, paxillin and FAK, which leads to disappearance of stress fibers and F-actin accumulation in the membrane ruffles (Han and Rubin, 1996; Troyer et al., 1996); 3) PKA-mediated modulation of Rho GTPase activity. PKA can phosphorylate RhoA at Ser¹⁸⁸ (Lang et al., 1996) and thus decrease Rho association with Rho kinase (Busca et al., 1998; Dong et al., 1998). PKA activation also increases interaction of RhoA with Rho-GDP dissociation inhibitor (Rho-GDI) and translocation of RhoA from the membrane to the cytosol (Lang et al., 1996; Qiao et al., 2003; Tamma et al., 2003). Thus, the overall effect of PKA on RhoA is downregulation of RhoA activity and stabilization of cortical actin cytoskeleton, which may promote EC barrier properties. Activation of PKC by phorbol esters induces specific cytoskeletal remodeling and exhibits barrier-disruptive effect on macrovascular EC, however it promotes barrier-protective response in lung microvascular EC (Bogatcheva et al., 2003). In addition, recent studies demonstrate that monolayer permeability changes are differentially regulated by PKC isoenzymes, suggesting that PKC alpha promotes endothelial barrier dysfunction and PKC delta enhances basal endothelial barrier function (Harrington et al., 2003). Further studies aimed at analysis of isoform-specific PKC activation will shed a light on the role of PKC isoforms in OxPAPC-induced cell signaling and endothelial cell function.

Although kinetics of OxPAPC-mediated intracellular signaling suggests receptor type of cellular response, specific receptor for OxPAPC has not been yet identified. Some, but not all, effects of OxPAPC, can be partially attenuated by platelet activating factor (PAF) receptor antagonists (Kadl et al., 2002; Leitinger et al., 1997), whereas PAF itself does not mimic OxPAPC effects (Leitinger et al., 1997). These observations suggest potential structural homology of putative OxPAPC receptor with PAF receptor.

In this study we also examined potential downstream cytoskeletal targets of OxPAPC-mediated signaling. Previous reports suggest, that oxidized LDL may cause Rho-mediated stress fiber formation, robust MLC phosphorylation in endothelial cells and actin polymerization in platelets (Essler et al., 1999; Maschberger et al., 2000). Results of our study suggest that OxPAPC did not increase the levels of MLC phosphorylation in HPAEC. Moreover, site-specific analysis of MYPT1 phosphorylation sites, Thr⁶⁸⁶ and Thr⁸⁵⁰, which are specific sites for phosphorylation by Rho-associated kinase (Carbajal et al., 2000; Velasco et al., 2002), showed no changes in phosphorylation after OxPAPC treatment. These results clearly indicate that OxPAPC treatment does not increase MLC phosphorylation, which is tightly linked to actomyosin contraction in HPAEC (Dudek and Garcia, 2001). However, we observed increases in phosphorylation of cofilin, an actin binding protein involved in regulation of actin polymerization. Non-phosphorylated cofilin binds actin monomers and prevents actin polymerization, whereas cofilin phosphorylation abolishes cofilin-actin interaction and thus promotes actin polymerization (Chen et al., 2000; Cooper and Schafer, 2000). Thus, our results strongly suggest involvement of OxPAPC in HPAEC actin remodeling via cofilin phosphorylation, and further studies are underway to more precisely characterize human pulmonary EC remodeling induced by OxPAPC. Consistent with proposed cytoskeletal effects of OxPAPC, we demonstrate that OxPAPC challenge also induced phosphorylation of focal adhesion proteins paxillin and focal adhesion kinase (FAK). Paxillin is a multi-domain adapter focal adhesion protein containing binding sites for various signaling molecules and structural proteins (Birge et al., 1993; Turner et al., 1990; Turner and Miller, 1994). Paxillin facilitates signal transduction from extracellular matrix and receptor-dependent agonists by recruiting specific molecules to focal adhesions, and paxillin phosphorylation by FAK at Tyr¹¹⁸ is important for determining its binding partners (Bellis et al., 1995; Schaller and Parsons, 1995; Turner, 1998). In turn, FAK autophosphorylation and phosphorylation by other tyrosine kinases, such as p60Src is a major mechanism for regulation of FAK catalytic activity and interaction with binding partners (Parsons et al., 2000; Schaller, 2001). Therefore, increased FAK and paxillin tyrosine phosphorylation in OxPAPC-stimulated HPAEC and its attenuation by specific P60Src inhibitor, PP-2, suggest effects of OxPAPC on focal adhesion remodeling, which may be mediated by p60Src and FAK.

In summary, this study provides for the first time comprehensive analysis of OxPAPC-mediated signaling and suggests potential effects of oxidized phospholipids on specific gene expression and cytoskeletal remodeling in EC from pulmonary circulation. We described OxPAPC-mediated activation of MAP kinase cascades and PKC and PKA catalytic activities in human pulmonary endothelium. We demonstrated activation of specific regulatory proteins, cofilin, paxillin and FAK, involved in remodeling of actin cytoskeleton and cell focal adhesions. Taken together with stimulatory effects of OxPAPC on tissue factor expression and monocyte adhesion to endothelium, previously described in systemic circulation (Bochkov et al., 2002b; Leitinger et al., 1997; Subbanagounder et al., 2000), our data suggest a novel role for oxidized phospholipids in pulmonary circulation related to modulation of lung inflammatory response and EC cytoskeletal changes.

### REFERENCES

Adams J C, Schwartz M A. 2000. Stimulation of fascin spikes by thrombospondin-1 is mediated by the GTPases Rac and Cdc42. J Cell Biol. 150:807-22.
Adkins, W. K., J. W. Barnard, T. M. Moore, R. C. Allison, V. R. Prasad, and A. E. Taylor. 1993. Adenosine prevents PMA-induced lung injury via an A2 receptor mechanism. J Appl Physiol. 74:982-8.
Bellis, S. L., J. T. Miller, and C. E. Turner. 1995. Characterization of tyrosine phosphorylation of paxillin in vitro by focal adhesion kinase. J Biol. Chem. 270:17437-41.
Berliner, J. A., M. C. Territo, A. Sevanian, S. Ramin, J. A. Kim, B. Bamshad, M.
Esterson, and A. M. Fogelman. 1990. Minimally modified low density lipoprotein stimulates monocyte endothelial interactions. J Clin Invest. 85:1260-6.
Bird M M, Lopez-Lluch G, Ridley A J, Segal A W. 2003. Effects of microinjected small GTPases on the actin cytoskeleton of human neutrophils. J Anat. 203:379-89.
Birge, R. B., J. E. Fajardo, C. Reichman, S. E. Shoelson, Z. Songyang, L. C. Cantley, and H. Hanafusa. 1993. Identification and characterization of a high-affinity interaction between v-Crk and tyrosine-phosphorylated paxillin in CT10-transformed fibroblasts. Mol Cell Biol. 13:4648-56.
Birukov, K. G., J. R. Jacobson, A. A. Flores, S. Q. Ye, A. A. Birukova, A. D. Verin, and J. G. Garcia. 2003. Magnitude-dependent regulation of pulmonary endothelial cell barrier function by cyclic stretch. Am J Physiol Lung Cell Mol Physiol.
Birukov K G, Leitinger N, Bochkov V N, Garcia J G. 2004. Signal transduction pathways activated in human pulmonary endothelial cells by OxPAPC, a bioactive component of oxidized lipoproteins. Microvasc Res. 67:18-28.
Birukova A A, Smurova K, Birukov K G, Kaibuchi K, Garcia J G N, Verin A D. 2004. Role of Rho GTPases in thrombin-induced lung vascular endothelial cells barrier dysfunction. Microvasc Res. 67:64-77.
Birukov K G, Birukova A A, Dudek S M, Verin A D, Crow M T, Zhan X, DePaola N, Garcia J G. 2002. Shear stress-mediated cytoskeletal remodeling and cortactin translocation in pulmonary endothelial cells. Am J Respir Cell Mol Biol. 26:453-64.
Birukova A A, Birukov K G, Smurova K, Adyshev D M, Kaibuchi K, Alieva I, Garcia J G, Verin A D. 2004. Novel role of microtubules in thrombin-induced endothelial barrier dysfunction. FASEB J. in press
Bochkov, V. N., A. Kadl, J. Huber, F. Gruber, B. R. Binder, and N. Leitinger. 2002a. Protective role of phospholipid oxidation products in endotoxin-induced tissue damage. Nature. 419:77-81.
Bochkov, V. N., D. Mechtcheriakova, M. Lucema, J. Huber, R. Malli, W. F. Graier, E. Hofer, B. R. Binder, and N. Leitinger. 2002b. Oxidized phospholipids stimulate tissue factor expression in human endothelial cells via activation of ERK/EGR-1 and Ca(++)/NFAT. Blood. 99:199-206.
Bogatcheva, N. V., J. G. Garcia, and A. D. Verin. 2002. Molecular mechanisms of thrombin-induced endothelial cell permeability. Biochemistry (Mosc). 67:75-84.
Bogatcheva, N. V., A. D. Verin, P. Wang A. A. Birukova, K. G. Birukov, T. Mirzopoyazova, D. M. Adyshev, E. T. Chiang, M. T. Crow, and J. G. Garcia. 2003. Phorbol esters increase MLC phosphorylation and actin remodeling in bovine lung endothelium without increased contraction. Am J Physiol Lung Cell Mol Physiol. 285:L415-26.
Borisy G G, Svitkina T M. 2000. Actin machinery: pushing the envelope. Curr Opin Cell Biol. 12:104-12.
Busca, R., C. Bertolotto, P. Abbe, W. Englaro, T. Ishizaki, S. Narumiya, P. Boquet, J. P. Ortonne, and R. Ballotti. 1998. Inhibition of Rho is required for cAMP-induced melanoma cell differentiation. Mol Biol Cell. 9:1367-78.
Carbajal, J. M., M. L. Gratrix, C. H. Yu, and R. C. Schaeffer, Jr. 2000. ROCK mediates thrombin's endothelial barrier dysfunction. Am J Physiol Cell Physiol. 279:C195-204.
Chabot, F., J. A. Mitchell, J. M. Gutteridge, and T. W. Evans. 1998. Reactive oxygen species in acute lung injury. Eur Respir J. 11:745-57.
Chen, H., B. W. Bernstein, and J. R. Bamburg. 2000. Regulating actin-filament dynamics in vivo. Trends Biochem Sci. 25:19-23.
Chetham, P. M., H. A. Guldemeester, N. Mons, G. H. Brough, J. P. Bridges, W. J. Thompson, and T. Stevens. 1997. Ca(2+)-inhibitable adenylyl cyclase and pulmonary microvascular permeability. Am J. Physiol. 273:L22-30.
Cooper, J. A., and D. A. Schafer. 2000. Control of actin assembly and disassembly at filament ends. Curr Opin Cell Biol. 12:97-103.
Dong, J. M., T. Leung, E. Manser, and L. Lim. 1998. cAMP-induced morphological changes are counteracted by the activated RhoA small GTPase and the Rho kinase ROKalpha. J Biol. Chem. 273:22554-62.
Dudek, S. M., and J. G. Garcia. 2001. Cytoskeletal regulation of pulmonary vascular permeability. J Appl Physiol. 91:1487-500.
Emmett M, Fowler A A, Hyers T M, Crowle A J. 1987. Crossed immunoelectrophoretic analysis of ARDS lavage proteins. Proc Soc Exp Biol Med. 184:83-91.
Essler, M., M. Retzer, M. Bauer, J. W. Heemskerk, M. Aepfelbacher, and W. Siess. 1999. Mildly oxidized low density lipoprotein induces contraction of human endothelial cells through activation of Rho/Rho kinase and inhibition of myosin light chain phosphatase. J Biol. Chem. 274:30361-4.
Essler, M., J. M. Staddon, P. C. Weber, and M Aepfelbacher. 2000. Cyclic AMP blocks bacterial lipopolysaccharide induced myosin light chain phosphorylation in endothelial cells through inhibition of Rho/Rho kinase signaling [In Process Citation]. J Immunol. 164:6543-9. Freyssinet, J. M., F. Toti, B. Hugel, C. Gidon-Jeangirard, C. Kunzelmann, M. C. Martinez, and D. Meyer. 1999. Apoptosis in vascular disease. Thromb Haemost. 82:727-35.
Garcia, J. G., H. W. Davis, and C. E. Patterson. 1995. Regulation of endothelial cell gap formation and barrier dysfunction: role of myosin light chain phosphorylation. J Cell Physiol. 163:510-22.
Garcia, J. G., V. Lazar, L. I. Gilbert-McClain, P. J. Gallagher, and A. D. Verin. 1997. Myosin light chain kinase in endothelium: molecular cloning and regulation. Am J Respir Cell Mol. Biol. 16:489-94.
Garcia, J. G., F. Liu, A. D. Verin, A. Birukova, M. A. Dechert, W. T. Gerthoffer, J. R. Bamberg, and D. English. 2001. Sphingosine 1-phosphate promotes endothelial cell barrier integrity by Edg-dependent cytoskeletal rearrangement. J Clin Invest. 108:689-701.
Goggel R, Winoto-Morbach S, Vielhaber G, Imai Y, Lindner K, Brade L, Brade H, Ehlers S, Slutsky A S, Schutze S, Gulbins E, Uhlig S. 2004. PAF-mediated pulmonary edema: a new role for acid sphingomyelinase and ceramide. Nat Med. 10:155-60.
Gutkind, J. S. 1998. Cell growth control by G protein-coupled receptors: from signal transduction to signal integration. Oncogene. 17:1331-42.
Han, J. D., and C. S. Rubin. 1996. Regulation of cytoskeleton organization and paxillin dephosphorylation by cAMP. Studies on murine Y1 adrenal cells. J Biol. Chem. 271:29211-5.
Harrington, E. O., J. L. Brunelle, C. J. Shannon, E. S. Kim, K. Mennella, and S. Rounds. 2003. Role of protein kinase C isoforms in rat epididymal microvascular endothelial barrier function. Am J Respir Cell Mol. Biol. 28:626-36. Hastie, L. E., W. F. Patton, H. B. Hechtman, and D. Shepro. 1997. H2O₂-induced filamin redistribution in endothelial cells is modulated by the cyclic AMP-dependent protein kinase pathway. J Cell Physiol. 172:373-81.
Holme, P. A., N. O. Solum, F. Brosstad, M. Roger, and M. Abdelnoor. 1994. Demonstration of platelet-derived microvesicles in blood from patients with activated coagulation and fibrinolysis using a filtration technique and western blotting. Thromb Haemost. 72:666-71.
Huber J, Vales A, Mitulovic G, Blumer M, Schmid R, Witztum J L, Binder B R, Leitinger 2002. N. Oxidized membrane vesicles and blebs from apoptotic cells contain biologically active oxidized phospholipids that induce monocyte-endothelial interactions. Arterioscler Thromb Vasc Biol. 22:101-7
Kaibuchi K, Kuroda S, Amano M. 1999. Regulation of the cytoskeleton and cell adhesion by the Rho family GTPases in mammalian cells. Annu Rev Biochem. 68:459-86.
Kaibuchi K, Kuroda S, Fukata M, Nakagawa M. 1999b. Regulation of cadherin-mediated cell-cell adhesion by the Rho family GTPases. Curr Opin Cell Biol. 11:591-6.
Kadl, A., J. Huber, F. Gruber, V. N. Bochkov, B. R. Binder, and N. Leitinger. 2002. Analysis of inflammatory gene induction by oxidized phospholipids in vivo by quantitative real-time RT-PCR in comparison with effects of LPS. Vascul Pharmacol. 38:219-27.
Lang, J. D., P. J. McArdle, P. J. O'Reilly, and S. Matalon. 2002. Oxidant-antioxidant balance in acute lung injury. Chest. 122:314S-320S.
Lang, P., F. Gesbert, M. Delespine-Carmagnat, R. Stancou, M. Pouchelet, and J. Bertoglio. 1996. Protein kinase A phosphorylation of RhoA mediates the morphological and functional effects of cyclic AMP in cytotoxic lymphocytes. Embo J. 15:510-9.
Leitinger, N., T. R. Tyner, L. Oslund, C. Rizza, G. Subbanagounder, H. Lee, P. T. Shih, N. Mackman, G. Tigyi, M. C. Territo, J. A. Berliner, and D. K. Vora. 1999. Structurally similar oxidized phospholipids differentially regulate endothelial binding of monocytes and neutrophils. Proc Natl Acad Sci USA. 96:12010-5.
Leitinger N, Watson A D, Faull K F, Fogelman A M, Berliner J A. 1997. Monocyte binding to endothelial cells induced by oxidized phospholipids present in minimally oxidized low density lipoprotein is inhibited by a platelet activating factor receptor antagonist. Adv Exp Med Biol. 433:379-82.
Levy Y, Ronen D, Bershadsky A D, Zick Y. 2003. Sustained induction of ERK, protein kinase B, and p70 S6 kinase regulates cell spreading and formation of F-actin microspikes upon ligation of integrins by galectin-8, a mammalian lectin. J Biol Chem. 278:14533-42.
Li, D., L. Liu, H. Chen, T. Sawamura, S. Ranganathan, and J. L. Mehta. 2003. LOX-1 mediates oxidized low-density lipoprotein-induced expression of matrix metalloproteinases in human coronary artery endothelial cells. Circulation. 107:612-7.
Li, D., B. Yang, and J. L. Mehta. 1998. Ox-LDL induces apoptosis in human coronary artery endothelial cells: role of PKC, PTK, bcl-2, and Fas. Am J Physiol. 275:H568-76.
Liu, F., A. D. Verin, T. Borbiev, and J. G. Garcia. 2001. Role of cAMP-dependent protein kinase A activity in endothelial cell cytoskeleton rearrangement. Am J Physiol Lung Cell Mol Physiol. 280:L1309-17.
Lum, H., and A. B. Malik. 1996. Mechanisms of increased endothelial permeability. Can J Physiol Pharmacol. 74:787-800.
Luttrell, L. M., Y. Daaka, and R. J. Lefkowitz. 1999. Regulation of tyrosine kinase cascades by G-protein-coupled receptors. Curr Opin Cell Biol. 11: 177-83.
Machesky L M, Insall R H. 1999. Signaling to actin dynamics. J Cell Biol. 146:267-72.
Mallat, Z., H. Benamer, B. Hugel, J. Benessiano, P. G. Steg, J. M. Freyssinet, and A. Tedgui. 2000. Elevated levels of shed membrane microparticles with procoagulant potential in the peripheral circulating blood of patients with acute coronary syndromes. Circulation. 101:841-3.
Maschberger, P., M. Bauer, J. Baumann-Siemons, K. J. Zangl, E. V. Negrescu, A. J. Reininger, and W. Siess. 2000. Mildly oxidized low density lipoprotein rapidly stimulates via activation of the lysophosphatidic acid receptor Src family and Syk tyrosine kinases and Ca2+ influx in human platelets. J Biol. Chem. 275:19159-66.
Matot, I., Y. Manevich, A. B. Al-Mehdi, C. Song, and A. B. Fisher. 2003. Fluorescence imaging of lipid peroxidation in isolated rat lungs during nonhypoxic lung ischemia. Free Radic Biol Med. 34:785-90.
Matsuoka, Y., C. A. Hughes, and V. Bennett. 1996. Adducin regulation. Definition of the calmodulin-binding domain and sites of phosphorylation by protein kinases A and C. J Biol. Chem. 271:25157-66.
Mine, S., T. Tabata, Y. Wada, T. Fujisaki, T. Iida, N. Noguchi; E. Niki, T. Kodama, and Y. Tanaka. 2002. Oxidized low density lipoprotein-induced LFA-1-dependent adhesion and transendothelial migration of monocytes via the protein kinase C pathway. Atherosclerosis. 160:281-8.
Nakamura, T., P. M. Henson, and R. C. Murphy. 1998. Occurrence of oxidized metabolites of arachidonic acid esterified to phospholipids in murine lung tissue. Anal Biochem. 262:23-32.
Napoli, C., O. Quehenberger, F. De Nigris, P. Abete, C. K. Glass, and W. Palinski. 2000. Mildly oxidized low density lipoprotein activates multiple apoptotic signaling pathways in human coronary cells. Faseb J. 14:1996-2007.
Nieuwland, R., R. J. Berckmans, R. C. Rotteveel-Eijkman, K. N. Maquelin, K. J. Roozendaal, P. G. Jansen, K. ten Have, L. Eijsman, C. E. Hack, and A. Sturk. 1997. Cell-derived microparticles generated in patients during cardiopulmonary bypass are highly procoagulant. Circulation. 96:3534-41.
Parsons, J. T., K. H. Martin, J. K. Slack, J. M. Taylor, and S. A. Weed. 2000. Focal adhesion kinase: a regulator of focal adhesion dynamics and cell movement. Oncogene. 19:5606-13.
Patel, K. D., G. A. Zimmerman, S. M. Prescott, and T. M. McIntyre. 1992. Novel leukocyte agonists are released by endothelial cells exposed to peroxide. J Biol. Chem. 267:15168-75.
Patterson, C. E., H. W. Davis, K. L. Schaphorst, and J. G. Garcia. 1994. Mechanisms of cholera toxin prevention of thrombin- and PMA-induced endothelial cell barrier dysfunction. Microvasc Res. 48:212-35.
Patterson, C. E., H. Lum, K. L. Schaphorst, A. D. Verin, and J. G. Garcia. 2000. Regulation of endothelial barrier function by the cAMP-dependent protein kinase. Endothelium. 7:287-308.
Porter, A. C., and R. R. Vaillancourt. 1998. Tyrosine kinase receptor-activated signal transduction pathways which lead to oncogenesis. Oncogene. 17:1343-52.
Qiao, J., F. Huang, and H. Lum. 2003. PKA inhibits RhoA activation: a protection mechanism against endothelial barrier dysfunction. Am J Physiol Lung Cell Mol Physiol. 284:L972-80.
Ridley A J. Rho family proteins: coordinating cell responses. 2001. Trends Cell Biol. 11:471-7.
Schaller, M. D. 2001. Biochemical signals and biological responses elicited by the focal adhesion kinase. Biochim Biophys Acta. 1540:1-21.
Schaller, M. D., and J. T. Parsons. 1995. pp125FAK-dependent tyrosine phosphorylation of paxillin creates a high-affinity binding site for Crk. Mol Cell Biol. 15:2635-45.
Schaphorst, K. L., F. M. Pavalko, C. E. Patterson, and J. G. Garcia. 1997. Thrombin-mediated focal adhesion plaque reorganization in endothelium: role of protein phosphorylation. Am J Respir Cell Mol. Biol. 17:443-55.
Shikata, Y., K. G. Birukov, and J. G. Garcia. 2003a. S1P induces FA remodeling in human pulmonary endothelial cells: role of Rac, GIT1, FAK and paxillin. J Appl Physiol. 94:1193-1203.
Shikata, Y., K. G. Birukov, and J. G. Garcia. 2003b. Site-specific FAK phosphorylation is involved in sphingosine-1 phosphate- and thrombin-induced focal adhesion remodeling: role of Src, GIT1 and GIT2. Faseb J: In press.
Steinberg, D., S. Parthasarathy, T. E. Carew, J. C. Khoo, and J. L. Witztum. 1989. Beyond cholesterol. Modifications of low-density lipoprotein that increase its atherogenicity. N Engl J. Med. 320:915-24.
Subbanagounder, G., N. Leitinger, D. C. Schwenke, J. W. Wong, H. Lee, C. Rizza, A. D. Watson, K. F. Faull, A. M. Fogelman, and J. A. Berliner. 2000. Determinants of bioactivity of oxidized phospholipids. Specific oxidized fatty acyl groups at the sn-2 position. Arterioscler Thromb Vasc Biol. 20:2248-54.
Subbanagounder G, Leitinger N, Shih P T, Faull K F, Berliner J A. 1999. Evidence that phospholipid oxidation products and/or platelet-activating factor play an important role in early atherogenesis: in vitro and In vivo inhibition by WEB 2086. Circ Res. 85:311-8.
Tamma, G., E. Klussmann, G. Procino, M. Svelto, W. Rosenthal, and G. Valenti. 2003. cAMP-induced AQP2 translocation is associated with RhoA inhibition through RhoA phosphorylation and interaction with RhoGDI. J Cell Sci. 116:1519-25.
Troyer, D. A., A. Bouton, R. Bedolla, and R. Padilla. 1996. Tyrosine phosphorylation of focal adhesion kinase (p125FAK): regulation by cAMP and thrombin in mesangial cells. J Am Soc Nephrol. 7:415-23.
Turner, C. E. 1998. Paxillin. Int J Biochem Cell Biol. 30:955-9.
Turner, C. E. 2000. Paxillin and focal adhesion signalling. Nat Cell Biol. 2:E231-6.
Turner, C. E., J. R. Glenney, Jr., and K. Burridge. 1990. Paxillin: a new vinculin-binding protein present in focal adhesions. J Cell Biol. 111:1059-68.
Turner, C. E., and J. T. Miller. 1994. Primary sequence of paxillin contains putative SH2 and SH3 domain binding motifs and multiple LIM domains: identification of a vinculin and pp125Fak-binding region. J Cell Sci. 107:1583-91.
Turner C E, West K A, Brown M C. 2001. Paxillin-ARF GAP signaling and the cytoskeleton. Curr Opin Cell Biol. 13:593-9.
Uhlson C, Harrison K, Allen C B, Ahmad S, White C W, Murphy R C. 2002. Oxidized phospholipids derived from ozone-treated lung surfactant extract reduce macrophage and epithelial cell viability. Chem Res Toxicol. 15:896-906.
van Nieuw Amerongen G P, van Delft S, Vermeer M A, Collard J G, van Hinsbergh V W. 2000. Activation of RhoA by thrombin in endothelial hyperpermeability: role of Rho kinase and protein tyrosine kinases. Circ Res. 87:335-40.
Velasco, G., C. Armstrong, N. Morrice, S. Frame, and P. Cohen. 2002. Phosphorylation of the regulatory subunit of smooth muscle protein phosphatase 1M at Thr850 induces its dissociation from myosin. FEBS Lett. 527:101-4.
Wallach, D., P. J. Davies, and I. Pastan. 1978. Cyclic AMP-dependent phosphorylation of filamin in mammalian smooth muscle. J Biol. Chem. 253:4739-45.
Watson, A. D., N. Leitinger, M. Navab, K. F. Faull, S. Horkko, J. L. Witztum, W. Palinski, D. Schwenke, R. G. Salomon, W. Sha, G. Subbanagounder, A. M. Fogelman, and J. A. Berliner. 1997. Structural identification by mass spectrometry of oxidized phospholipids in minimally oxidized low density lipoprotein that induce monocyte/endothelial interactions and evidence for their presence in vivo. J Biol. Chem. 272:13597-607.
Weed S A, Parsons J T. 2001. Cortactin: coupling membrane dynamics to cortical actin assembly. Oncogene. 20:6418-34.
Wood, L. G., P. G. Gibson, and M. L. Garg. 2003. Biomarkers of lipid peroxidation, airway inflammation and asthma. Eur Respir J. 21:177-86.
Yang, C. M., C. S. Chien, L. D. Hsiao, S. L. Pan, C. C. Wang, C. T. Chiu, and C. C. Lin. 2001. Mitogenic effect of oxidized low-density lipoprotein on vascular smooth muscle cells mediated by activation of Ras/Raf/MEK/MAPK pathway. Br J Pharmacol. 132:153141.
   Incorporation by Reference

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of enhancing endothelial cell barrier protective activity in a subject comprising:
administering to a subject an effective amount of oxidized phospholipids;
thereby enhancing the endothelial cell barrier protective activity in the subject.

2. The method of claim 1, wherein the phospholipids are phosphatidylserines, phosphatidylinositols, phosphatidylethanolamines, phosphatidylcholines or 1-Palmytoyl-2-Arachidonoyl-sn-Glycero-2-Phosphates.

3. The method of claim 1, wherein the phospholipids have unsaturated bonds.

4. The method of claim 1, wherein the phospholipids are arachidonic acid containing phospholipids.

5. The method of claim 1, wherein the phospholipids are sn-2-oxygenated.

6. The method of claim 1, wherein the phospholipids are not fragmented.

7. The method of claim 1, wherein the oxidized phospholipids are oxidized 1-palmitoyl-2-arachidonoyl-sn-glycero-3-phosphorylchloine (OxPAPC).

8. The method of claim 7, wherein the oxPAPCs are epoxyisoprostane-containing phospholipids.

9. The method of claim 8, wherein the oxPAPC is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

10. The method of claim 1, for the treatment of a subject having acute lung injury syndromes, sepsis, vascular leakage, edema, acute respiratory distress syndrome (ARDS) or acute inflammation.

11. A method of enhancing endothelial cell barrier protective activity in a subject comprising:
administering to a subject an effective amount of epoxyisoprostane-containing phospholipids;
thereby enhancing the endothelial cell barrier protective activity in the subject.

12. The method of claim 11, wherein the epoxyisoprostane-containing phospholipids are 1-palmitoyl-2-(5,6-epoxyisoprostane E2)-sn-glycero-3-phosphocholines (5,6-PEIPC), 1-palmitoyl-2-(epoxycyclopentenone)-sn-glycero-3-phosphorylcholines (PECPC) or 1-palmitoyl-2-(epoxyisoprostane E2)-sn-glycero-4-phosphocholines (PEIPC).

13. A method of treating a subject having an acute lung injury comprising;
administering to a subject an effective amount of oxidized phospholipids;
thereby treating the acute lung injury in the subject.

14. The method of claim 13, wherein the phospholipids are phosphatidylserines, phosphatidylinositols, phosphatidylethanolamines, phosphatidylcholines or 1-Palmytoyl-2-Arachidonoyl-sn-Glycero-2-Phosphates.

15. The method of claim 13, wherein the phospholipids have unsaturated bonds.

16. The method of claim 13, wherein the phospholipids are arachidonic acid containing phospholipids.

17. The method of claim 13, wherein the phospholipids are sn-2-oxygenated.

18. The method of claim 13, wherein the phospholipids are not fragmented.

19. The method of claim 13, wherein the oxidized phospholipids are oxidized 1-palmitoyl-2-arachidonoyl-sn-glycero-3-phosphorylchloine (OxPAPC).

20. The method of claim 19, wherein the oxPAPCs are epoxyisoprostane-containing phospholipids.

21. The method of claim 20, wherein the oxPAPC is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

22. A method of treating a subject having an acute lung injury comprising;
administering to a subject an effective amount of epoxyisoprostane-containing phospholipids;
thereby treating the acute lung injury in the subject.

23. The method of claim 22, wherein the epoxyisoprostane-containing phospholipids are 1-palmitoyl-2-(5,6-epoxyisoprostane E2)-sn-glycero-3-phosphocholines (5,6-PEIPC), 1-palmitoyl-2-(epoxycyclopentenone)-sn-glycero-3-phosphorylcholines (PECPC) or 1-palmitoyl-2-(epoxyisoprostane E2)-sn-glycero-4-phosphocholines (PEIPC).

24. A method of treating a subject having sepsis comprising;
administering to a subject an effective amount of oxidized phospholipids;
thereby treating sepsis in the subject.

25. The method of claim 24, wherein the phospholipids are phosphatidylserines, phosphatidylinositols, phosphatidylethanolamines, phosphatidylcholines or 1-Palmytoyl-2-Arachidonoyl-sn-Glycero-2-Phosphates.

26. The method of claim 24, wherein the phospholipids have unsaturated bonds.

27. The method of claim 24, wherein the phospholipids are arachidonic acid containing phospholipids.

28. The method of claim 24, wherein the phospholipids are sn-2-oxygenated.

29. The method of claim 24, wherein the phospholipids are not fragmented.

30. The method of claim 24, wherein the oxidized phospholipids are oxidized 1-palmitoyl-2-arachidonoyl-sn-glycero-3-phosphorylchloine (OxPAPC).

31. The method of claim 30, wherein the oxPAPCs are epoxyisoprostane-containing phospholipids.

32. The method of claim 31, wherein the oxPAPC is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero4-phosphocholine (PEIPC).

33. A pharmaceutical composition comprising oxidized phospholipids and a pharmaceutically active carrier.

34. The pharmaceutical composition of claim 33, wherein the phospholipids are phosphatidylserines, phosphatidylinositols, phosphatidylethanolamines, phosphatidylcholines or 1-Palmytoyl-2-Arachidonoyl-sn-Glycero-2-Phosphates.

35. The pharmaceutical composition of claim 24, wherein the phospholipids have unsaturated bonds.

36. The pharmaceutical composition of claim 24, wherein the phospholipids are arachidonic acid containing phospholipids.

37. The pharmaceutical composition of claim 24, wherein the phospholipids are sn-2-oxygenated.

38. The pharmaceutical composition of claim 24, wherein the phospholipids are not fragmented.

39. The pharmaceutical composition of claim 24, wherein the oxidized phospholipids are oxidized 1-palmitoyl-2-arachidonoyl-sn-glycero-3-phosphorylchloine (OxPAPC).

40. The pharmaceutical composition of claim 30, wherein the oxPAPCs are epoxyisoprostane-containing phospholipids.

41. The pharmaceutical composition of claim 31, wherein the oxPAPC is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).

42. A kit for the treatment of acute lung injury syndromes, sepsis, vascular leakage, edema, acute respiratory distress syndrome (ARDS) or acute inflammation comprising oxidized phospholipids and instructions for use.

43. The kit of claim 42, wherein the phospholipids are phosphatidylserines, phosphatidylinositols, phosphatidylethanolamines, phosphatidylcholines or 1-Palmytoyl-2-Arachidonoyl-sn-Glycero-2-Phosphates.

44. The kit of claim 42, wherein the phospholipids have unsaturated bonds.

45. The kit of claim 42, wherein the phospholipids are arachidonic acid containing phospholipids.

46. The kit of claim 42, wherein the phospholipids are sn-2-oxygenated.

47. The kit of claim 42, wherein the phospholipids are not fragmented.

48. The kit of claim 42, wherein the oxidized phospholipids are oxidized 1-palmitoyl-2-arachidonoyl-sn-glycero-3-phosphorylchloine (OxPAPC).

49. The kit of claim 48, wherein the oxPAPCs are epoxyisoprostane-containing phospholipids.

50. The kit of claim 49, wherein the oxPAPC is 1-palmitoyl-2-(5,6-epoxyisoprostane E2)-sn-glycero-3-phosphocholine (5,6-PEIPC), 1-palmitoyl-2-(epoxycyclopentenone)-sn-glycero-3-phosphorylcholine (PECPC) or 1-palmitoyl-2-(epoxyisoprostane E2)-sn-glycero-4-phosphocholine (PEIPC).
